(19) United States
(12) Patent Application Publication
(10) Pub. No.: US 2018/0303605 A1
(43) Pub. Date: Oct. 25, 2018

(54) Tissue Fixation Systems, Delivery Tools, and Associated Kits and Methods

(71) Applicants: Shaun Tanner, West Lafayette, IN (US); Andrew Isch, Lafayette, IN (US); Nolan Xanh, West Lafayette, IN (US); Ali Ghamsari, West Lafayette, IN (US); Darin Schaeffer, Bloomington, IN (US); Jeffry Melsheimer, Springville, IN (US)

(72) Inventors: Shaun Tanner, West Lafayette, IN (US); Andrew Isch, Lafayette, IN (US); Nolan Xanh, West Lafayette, IN (US); Ali Ghamsari, West Lafayette, IN (US); Darin Schaeffer, Bloomington, IN (US); Jeffry Melsheimer, Springville, IN (US)

(21) Appl. No.: 15/961,265

(22) Filed: Apr. 24, 2018

Related U.S. Application Data
(60) Provisional application No. 62/489,597, filed on Apr. 25, 2017.

(51) Int. Cl. A61F 2/20

(52) U.S. Cl. CPC A61F 2/20; A61B 1/07; A61F 2220/0016; A61F 2220/0041

(57) ABSTRACT

Tissue fixation systems, delivery tools, kits that include a tissue fixation system, and methods of treatment that use a tissue fixation system are described herein. An example embodiment of a tissue fixation system includes an anchor, a set screw, and a tissue manipulator. The anchor has a shaft and a head attached to the shaft. The set screw is partially disposed within a passageway defined by the anchor and is moveable between a first position and a second position. The tissue manipulator is partially disposed within the passageway defined by the anchor and is removable from the anchor when the set screw is in the first position and is releasably attached to the anchor when the set screw is in the second position.

### RELATED APPLICATIONS

This application claims the benefit of United States Provisional Application No. 62/489,597, filed on Apr. 25, 2017. The disclosure of this related application is hereby incorporated into this disclosure by reference.

### FIELD

The disclosure relates generally to the field of medical devices. More particularly, the disclosure relates to tissue fixation systems, delivery tools, kits that include a tissue fixation system, and methods of treatment that use a tissue fixation system.

### BACKGROUND

Vocal cord paralysis can impair breathing, swallowing, and phonation. Various procedures have been developed to reposition immobile vocal cords into a phonatory position (adduction, abduction) to improve phonation, swallowing, and breathing in patients that have been inflicted with vocal cord paralysis. For example, some procedures reposition the vocal cord via arytenoid adduction or arytenopexy coupled with cricothyroid approximation. However, these procedures have not gained wide acceptance because of the complexity and invasiveness of the deep neck dissection required to establish access to the arytenoid(s). Another invasive procedure, thyroplasty, requires removal of a portion of the thyroid cartilage and implantation of a piece of material that acts as a shim to push the vocal fold to the midline. Once placed, though, the shim usually is not adjustable and the procedure itself is difficult to reverse. Injection laryngoplasty, which involves the injection of a material into the vocal folds to achieve a desired repositioning, offers a less invasive option. Unfortunately, though, multiple injections may be required to achieve a desired result and, for some patients, the results can be temporary.

Therefore, a need exists for new and useful approaches to vocal cord treatment.

### SUMMARY OF SELECTED EXAMPLE EMBODIMENTS

Various tissue fixation systems, delivery tools, kits that include a tissue fixation system, and methods of treatment that use a tissue fixation system are described herein.

An example tissue fixation system comprises an anchor, a set screw, and a tissue manipulator. The anchor has a shaft and a head attached to the shaft. The shaft has a shaft first end, a shaft second end, a shaft length that extends from the shaft first end to the shaft second end, and a shaft main body that defines a shaft outer surface, a shaft passageway that extends from the shaft first end to the shaft second end, and a shaft thread that extends from the shaft outer surface and along a portion of the shaft length. The head has a head first end attached to the shaft second end, a head second end, and a head main body that defines a head outer surface, a head inner surface, a head first passageway, a head second passageway, a head third passageway, and a head thread. The head first passageway has a first inside diameter, extends from the head first end toward the head second end to the head second passageway, and is in communication with the shaft passageway and the head second passageway. The head second passageway has a second inside diameter, extends from the head first passageway toward the head second end, and is in communication with the head first passageway and the head third passageway. The head third passageway has a third inside diameter, extends from the head second passageway to the head outer surface, and is in communication with the head second passageway. The first inside diameter is different than the second inside diameter. The second inside diameter is different than the third inside diameter. The head thread extends from the head inner surface and into the head third passageway. The set screw is partially disposed within the head third passageway and has a set screw first end, a set screw second end, a set screw length that extends from the set screw first end to the set screw second end, and a set screw main body that defines a set screw recess and a set screw thread that extends along a portion of the set screw length. The set screw recess extends from the set screw second end toward the set screw first end. The set screw thread is sized and configured to mate with the head thread. The set screw is moveable within the head third passageway between a first position and a second position. The tissue manipulator is partially disposed within the shaft passageway, the head first passageway, and the head second passageway and has a tissue manipulator shaft and a tissue manipulator coil attached to the tissue manipulator shaft. The tissue manipulator is removable from the anchor when the set screw is in the first position and is releasably attached to the anchor when the set screw is in the second position.

Another example tissue fixation system comprises an anchor, a set screw, and a tissue manipulator. The anchor has a shaft and a head attached to the shaft. The shaft has a shaft first end, a shaft second end, a shaft length that extends from the shaft first end to the shaft second end, and a shaft main body that defines a shaft outer surface, a shaft passageway that extends from the shaft first end to the shaft second end, and a shaft thread that extends from the shaft outer surface and along a portion of the shaft length. The head has a hexagonal cross-sectional configuration, head first end attached to the shaft second end, a head second end, and a head main body that defines a head outer surface, a head inner surface, a head first passageway, a head second passageway, a head third passageway, and a head thread. The head first passageway has a first inside diameter, extends from the head first end toward the head second end to the head second passageway, and is in communication with the shaft passageway and the head second passageway. The head second passageway has a head second passageway lengthwise axis, a second inside diameter, extends from the head first passageway toward the head second end, and is in communication with the head first passageway and the head third passageway. The head third passageway has a head third passageway lengthwise axis disposed at an angle about 90 degrees relative to the head second passageway lengthwise axis, a third inside diameter, extends from the head second passageway to the head outer surface, and is in communication with the head second passageway. The first inside diameter is different than the second inside diameter. The second inside diameter is different than the third inside diameter. The head thread extends from the head inner surface and into the head third passageway. The set screw is partially disposed within the head third passageway and has a set screw first end, a set screw second end, a set screw length that extends from the set screw first end to the set screw second end, and a set screw main body that defines a set screw recess and a set screw thread that extends along a portion of the set screw length. The set screw recess extends from the set screw second end toward the set screw first end. The set screw thread is sized and configured to mate with the head thread. The set screw is moveable within the head third passageway between a first position and a second position. The tissue manipulator is partially disposed within the shaft passageway, the head first passageway, and the head second passageway and has a tissue manipulator shaft and a tissue manipulator coil attached to the tissue manipulator shaft. The tissue manipulator is removable from the anchor when the set screw is in the first position and is releasably attached to the anchor when the set screw is in the second position.

Another example tissue fixation system comprises an anchor, a set screw, and a tissue manipulator. The anchor has a shaft and a head attached to the shaft. The shaft has a shaft first end, a shaft second end, a shaft length that extends from the shaft first end to the shaft second end, and a shaft main body that defines a shaft outer surface, a shaft passageway that extends from the shaft first end to the shaft second end, and a self-tapping shaft thread that extends from the shaft outer surface and along a portion of the shaft length. The head has a hexagonal cross-sectional configuration, head first end attached to the shaft second end, a head second end, and a head main body that defines a head outer surface, a head inner surface, a head first passageway, a head second passageway, a head third passageway, and a head thread. The head first passageway has a first inside diameter, extends from the head first end toward the head second end to the head second passageway, and is in communication with the shaft passageway and the head second passageway. The head second passageway has a head second passageway lengthwise axis, a second inside diameter, extends from the head first passageway to the head second end, and is in communication with the head first passageway and the head third passageway. The head third passageway has a head third passageway lengthwise axis disposed at an angle about 90 degrees relative to the head second passageway lengthwise axis, a third inside diameter, extends from the head second passageway to the head outer surface, and is in communication with the head second passageway. The first inside diameter is different than the second inside diameter. The second inside diameter is different than the third inside diameter. The head thread extends from the head inner surface and into the head third passageway. The set screw is partially disposed within the head third passageway and has a set screw first end, a set screw second end, a set screw length that extends from the set screw first end to the set screw second end, and a set screw main body that defines a set screw recess and a set screw thread that extends along a portion of the set screw length. The set screw recess extends from the set screw second end toward the set screw first end. The set screw thread is sized and configured to mate with the head thread. The set screw is moveable within the head third passageway between a first position and a second position. The tissue manipulator is partially disposed within the shaft passageway, the head first passageway, and the head second passageway and has a tissue manipulator shaft and a tissue manipulator coil attached to the tissue manipulator shaft. The tissue manipulator is removable from the anchor when the set screw is in the first position and is releasably attached to the anchor when the set screw is in the second position.

An example delivery tool comprises a delivery tool first end, a delivery tool second end, a delivery tool lengthwise axis that is centrally located through the delivery tool first end and the delivery tool second end, a delivery tool length, a delivery tool first outside diameter, a delivery tool second outside diameter, and a delivery tool main body that defines a delivery tool handle, a delivery tool shaft, and a delivery tool passageway. The delivery tool length extends from the delivery tool first end to the delivery tool second end. The delivery tool passageway extends from the delivery tool first end to the delivery tool second end and is sized and configured to receive a portion of a tissue manipulator.

An example kit comprises a tissue fixation system according to an embodiment; a delivery tool according to an embodiment; instructions for use; and a storage container.

An example method of treatment that uses a tissue fixation system comprises the steps of: creating an opening in the body over the vocal cord intended to be treated to access the thyroid cartilage; introducing a needle through the opening and the thyroid cartilage and into the arytenoid muscle; verifying placement of the needle; withdrawing the optical fiber while maintaining the position of the needle; introducing a tissue manipulator through the needle until it contacts the arytenoid muscle; applying torque on the tissue manipulator while applying an axial force toward the arytenoid muscle such that the tissue manipulator rotates and engages the arytenoid muscle; trimming the tissue manipulator between the control handle and the shaft second end to create a new tissue manipulator end; withdrawing the needle from the tissue and thyroid cartilage while maintaining position of the tissue manipulator; applying an axial force on the tissue manipulator to achieve a desired placement of vocal cord; introducing the new tissue manipulator end into a shaft passageway of an anchor; advancing the anchor over the tissue manipulator until the anchor contacts the thyroid cartilage and such that a portion of the tissue manipulator is disposed within the anchor; attaching the anchor to the thyroid cartilage; asking the patient to phonate; tightening the set screw onto the tissue manipulator while the portion of the tissue manipulator is disposed within a passageway of the head of the anchor; bending a portion of the tissue manipulator that extends from the anchor relative to a portion that is disposed through the shaft passageway; trimming the tissue manipulator; positioning the portion of the tissue manipulator that has been bent relative to the portion that is disposed through the shaft passageway within a portion of the head of the anchor; asking the patient to phonate to evaluate the position of the vocal cord and to determine whether adjustments are needed; closing the opening made in the body.

Another example method of treatment that uses a tissue fixation system comprises the steps of: creating an opening in the body over the vocal cord intended to be treated to access the thyroid cartilage; creating a window in the thyroid cartilage; introducing a needle through the window and into the arytenoid muscle; verifying placement of the needle; withdrawing the optical fiber while maintaining the position of the needle; introducing a tissue manipulator through the needle until it contacts the arytenoid muscle; applying torque on the tissue manipulator while applying an axial force toward the arytenoid muscle such that the tissue manipulator rotates and engages the arytenoid muscle; trimming the tissue manipulator between the control handle and the shaft second end to create a new tissue manipulator end; withdrawing the needle from the tissue and thyroid cartilage while maintaining position of the tissue manipulator; applying an axial force on the tissue manipulator to achieve a desired placement of vocal cord; asking the patient to phonate; creating a marking indicia on the shaft of the tissue manipulator where it exits the thyroid cartilage while holding the tissue manipulator at desired location; determining a suitable location to position an anchor relative to the window created in the thyroid cartilage; attaching the anchor to the thyroid cartilage; positioning the marking indicia on the tissue manipulator adjacent to the window; bending a portion of the tissue manipulator that extends from the window at the mark and relative to a portion that extends through the thyroid cartilage; positioning the bent portion of the tissue manipulator within a portion of the anchor; tightening the set screw to the tissue manipulator while the portion of the tissue manipulator is disposed within a portion of the anchor; trimming the tissue manipulator; asking the patient to phonate to evaluate the position of the vocal cord and to determine whether adjustments are needed; closing the opening made in the body.

Additional understanding of the example tissue fixation systems, delivery tools, kits that include a tissue fixation system, and methods of treatment that use a tissue fixation system can be obtained by review of the detailed description, below, and the appended drawings.

### BRIEF DESCRIPTION OF THE DRAWINGS

FIG. 1 is a perspective view of a first example tissue fixation system.

FIG. 2 is an exploded view of the tissue fixation system illustrated in FIG. 1.

FIG. 3 is a top view of the anchor of the tissue fixation system illustrated in FIG. 1.

FIG. 4 is a side view of the anchor illustrated in FIG. 3.

FIG. 5 is a top view of the set screw of the tissue fixation system illustrated in FIG. 1.

FIG. 6 is a side view of the set screw illustrated in FIG. 5.

FIG. 7 is a perspective view of the tissue manipulator of the tissue fixation system illustrated in FIG. 1.

FIG. 8 is a perspective view of a second example tissue fixation system.

FIG. 9 is an exploded view of the tissue fixation system illustrated in FIG. 8.

FIG. 10 is a top view of the anchor of the tissue fixation system illustrated in FIG. 8.

FIG. 11 is a side view of the anchor illustrated in FIG. 10.

FIG. 12 is a top view of the set screw of the tissue fixation system illustrated in FIG. 8.

FIG. 13 is a side view of the set screw illustrated in FIG. 12.

FIG. 14 is a perspective view of a third example tissue fixation system.

FIG. 15 is an exploded view of the tissue fixation system illustrated in FIG. 14.

FIG. 16 is a top view of the anchor of the tissue fixation system illustrated in FIG. 14.

FIG. 17 is a side view of the anchor illustrated in FIG. 16.

FIG. 18 is another side view of the anchor illustrated in FIG. 16.

FIG. 19 is a top view of the set screw of the tissue fixation system illustrated in FIG. 14.

FIG. 20 is a side view of the set screw illustrated in FIG. 19.

FIG. 21 is a perspective view of an example delivery tool used to implant a tissue fixation system.

FIG. 22 is a side view of the delivery tool illustrated in FIG. 21.

FIG. 23 is a magnified view of area I illustrated in FIG. 22.

FIG. 24 illustrates an example kit that includes a tissue fixation system.

FIG. 25 is a schematic illustration of an example method of treatment using a tissue fixation system.

FIG. 26 illustrates a superior view of the thyroid cartilage and the arytenoid muscle during performance of the method illustrated in FIG. 25. A needle is disposed through the thyroid cartilage and is partially disposed within the arytenoid muscle.

FIG. 27 illustrates a superior view of the thyroid cartilage and the arytenoid muscle during performance of the method illustrated in FIG. 25. A needle is disposed through the thyroid cartilage and is partially disposed within the arytenoid muscle.

FIG. 28 illustrates a superior view of the thyroid cartilage and the arytenoid muscle during performance of the method illustrated in FIG. 25. A needle and a tissue manipulator are disposed through the thyroid cartilage and are partially disposed within the arytenoid muscle.

FIG. 29 illustrates a superior view of the thyroid cartilage and the arytenoid muscle during performance of the method illustrated in FIG. 25. A needle and a tissue manipulator are disposed through the thyroid cartilage and are partially disposed within the arytenoid muscle. The tissue manipulator has been advanced into the arytenoid muscle.

FIG. 30 illustrates a superior view of the thyroid cartilage and the arytenoid muscle during performance of the method illustrated in FIG. 25. A tissue manipulator is disposed through the thyroid cartilage and partially disposed within the arytenoid muscle.

FIG. 31 illustrates a superior view of the thyroid cartilage and the arytenoid muscle during performance of the method illustrated in FIG. 25. A tissue manipulator is disposed through the thyroid cartilage and is partially disposed within the arytenoid muscle. The arytenoid muscle has been repositioned.

FIG. 32 illustrates a superior view of the thyroid cartilage and the arytenoid muscle during performance of the method illustrated in FIG. 25. An anchor of a tissue fixation system is being attached to the thyroid cartilage with a delivery tool. A tissue manipulator is disposed through the anchor and is partially disposed within the arytenoid muscle and the delivery tool.

FIG. 33 illustrates a superior view of the thyroid cartilage and the arytenoid muscle during performance of the method illustrated in FIG. 25. An anchor of a tissue fixation system is attached to the thyroid cartilage and a tissue manipulator is disposed through the anchor and is partially disposed within the arytenoid muscle. The set screw of the anchor has been releasably attached to the anchor such that the tissue manipulator is releasably attached to the anchor. The tissue manipulator has been bent such that a portion of the tissue manipulator is disposed within a passageway defined by the head of the anchor.

FIG. 34 illustrates a superior view of the thyroid cartilage and the arytenoid muscle during performance of the method illustrated in FIG. 25. An anchor of a tissue fixation system is attached to the thyroid cartilage and a tissue manipulator is disposed through the anchor and partially disposed within the arytenoid muscle. The tissue manipulator has been trimmed.

FIG. 35 is a schematic illustration of another example method of treatment that uses a tissue fixation system.

FIG. 36 illustrates a superior view of the thyroid cartilage and the arytenoid muscle during performance of the method illustrated in FIG. 35. A tissue manipulator is disposed through the thyroid cartilage and is partially disposed within the arytenoid muscle. The arytenoid muscle has been repositioned.

FIG. 37 illustrates a superior view of the thyroid cartilage and the arytenoid muscle during performance of the method illustrated in FIG. 35. An anchor of a tissue fixation system is attached to the thyroid cartilage and a tissue manipulator is disposed through the anchor and is partially disposed within the arytenoid muscle. The tissue manipulator has been bent such that a portion of the tissue manipulator is disposed within a passageway defined by the head of the anchor.

FIG. 38 illustrates a superior view of the thyroid cartilage and the arytenoid muscle during performance of the method illustrated in FIG. 35. An anchor of a tissue fixation system is attached to the thyroid cartilage and a tissue manipulator is disposed through the anchor and is partially disposed within the arytenoid muscle. The set screw of the anchor has been releasably attached to the anchor such that the tissue manipulator is releasably attached to the anchor.

### DETAILED DESCRIPTION

The following detailed description and the appended drawings describe and illustrate various example embodiments of tissue fixation systems, delivery tools, kits that include a tissue fixation system, and methods of treatment that use a tissue fixation system. The description and illustration of these examples are provided to enable one skilled in the art to make and use a tissue fixation system, a delivery tool, to make a kit that includes a tissue fixation system, and to practice a method of treatment using a tissue fixation system. They are not intended to limit the scope of the claims in any manner.

As used herein, the term “engaged” and grammatically related terms refer to a first element or feature being associated with, interconnected with, disposed in, or attached to a second element or feature such that the first element or feature can alter the position of the second element or feature when an outside force is applied on a portion of the first element or feature.

As used herein, the term “fixation” refers to the ability of a first device, element, or feature to maintain a second device, element, or feature in a particular position.

As used herein, the term “diameter” refers to the length of a straight line passing from side to side through the center of a body, element, or feature, and does not impart any structural configuration on the body, element, or feature.

As used herein, the phrase “arytenoid muscle” refers to the arytenoid muscle, the thyroarytenoid muscle, the cricoarytenoid muscle, and the arytenoid muscular process.

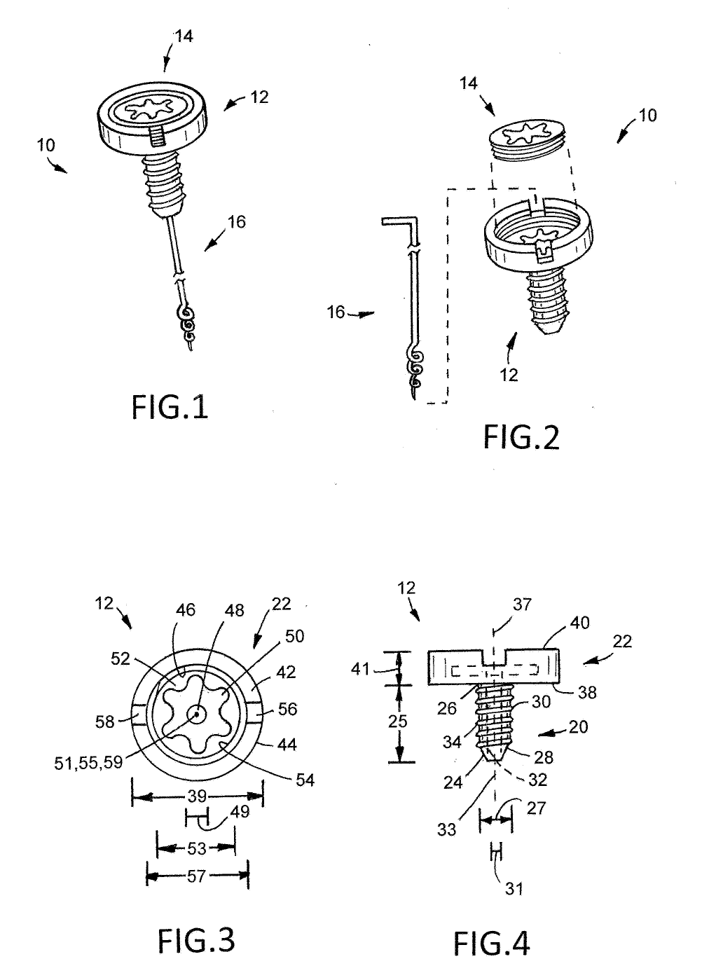

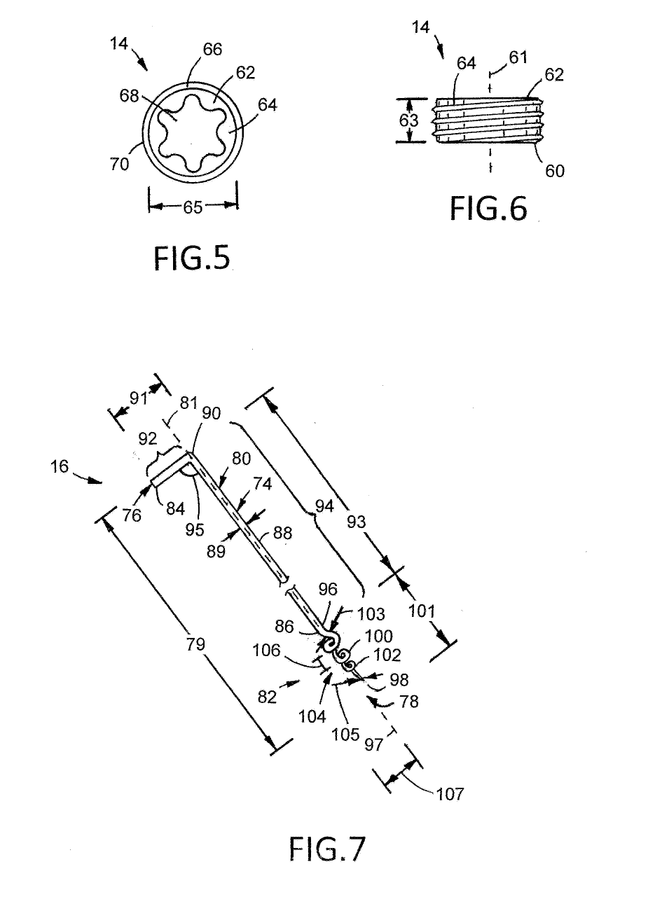

FIGS. 1, 2, 3, 4, 5, 6, and 7 illustrate a first example tissue fixation system 10. The tissue fixation system 10 includes an anchor 12, a set screw 14, and a tissue manipulator 16. In the illustrated embodiment, the set screw 14 is releasably attached to the anchor 12 and the tissue manipulator 16 is releasably attached to, and partially disposed through, the anchor 12.

The anchor 12 has a shaft 20 and a head 22 attached to the shaft 20. The shaft 20 has a shaft first end 24, a shaft second end 26, a shaft length 25 that extends from the shaft first end 24 to the shaft second end 26, a shaft outside diameter 27, and a shaft main body 28 that defines a shaft outer surface 30, a shaft passageway 32, and a shaft thread 34. The shaft passageway 32 extends from the shaft first end 24 to the shaft second end 26 and has a shaft passageway inside diameter 31, a shaft passageway lengthwise axis 33 that is centrally located through the shaft passageway, and a circular cross-sectional configuration taken along a plane that is disposed orthogonally to the shaft passageway lengthwise axis 33. The shaft passageway inside diameter 31 is measured along an axis that is disposed orthogonally to the shaft passageway lengthwise axis 33. The shaft passageway 32 is sized and configured to receive a portion of the tissue manipulator 16. The shaft thread 34 extends from the shaft outer surface 30, away from the shaft passageway lengthwise axis 33, and along a portion of the shaft length 25. In the illustrated embodiment, the shaft thread 34 is self-tapping.

While the shaft passageway 32 has been illustrated as having a cross-sectional configuration taken along a plane that is disposed orthogonally to the shaft passageway lengthwise axis 33 that is circular, a shaft passageway can have any suitable cross-sectional configuration taken along a plane that is disposed orthogonally to the shaft passageway lengthwise axis. Selection of a suitable cross-sectional configuration for a shaft passageway can be based on various considerations, including the cross-sectional configuration of the material that forms a tissue manipulator. Examples of cross-sectional configurations considered suitable for a shaft passageway include those that are circular, rectangular, square, hexagonal, and any other cross-sectional configuration considered suitable for a particular embodiment.

While the shaft thread 34 has been illustrated as self-tapping and as extending along a portion of the shaft length 25, a shaft thread can have any suitable structural arrangement and extend along any suitable portion of a shaft length. Selection of a suitable structural arrangement for a shaft thread and of a suitable portion of a shaft length for a shaft thread to extend can be based on various considerations, including the structural arrangement of the material to which an anchor is intended to be attached. Examples of structural arrangements considered suitable for a shaft thread include those that are self-tapping, V threads, American National threads, unified threads, Whitworth threads, square threads, acme threads, buttress threads, knuckle threads, single start threads, double start threads, triple start threads, self-tapping threads, threads that have any suitable number of flutes, and any other structural arrangement considered suitable for a particular embodiment. Examples of portions of a shaft length for a shaft thread to extend include along an entire shaft length, along a portion of a shaft length, from a shaft first end to a shaft second end, between a shaft first end to a shaft second end, from a shaft first end toward a shaft second end, from a shaft second end toward a shaft first end, and any other length considered suitable for a particular embodiment.

The head 22 has a head first end 38 attached to the shaft second end 26, a head second end 40, a head lengthwise axis 37 that is centrally located through the head first end 38 and the head second end 40, a head outside diameter 39, a head length 41 that extends from the head first end 38 to the head second end 40, and a head main body 42 that defines a head outer surface 44, a head inner surface 46, a head first passageway 48, a head second passageway 50, a head third passageway 52, a head thread 54, a head first notch 56, and a head second notch 58. The head length 41 is less than the shaft length 25. The head outside diameter 39 is greater than the shaft outside diameter 27. In the illustrated embodiment, the head main body 42 has a hollow circular cross-sectional configuration taken along a plane that is disposed orthogonally to the head lengthwise axis 37.

The head first passageway 48 has a head first passageway inside diameter 49, a head first passageway lengthwise axis 51 that is centrally located through the head first passageway 48, a head first passageway 48 cross-sectional configuration, extends from the head first end 38 toward the head second end 40 to the head second passageway 50, and is in communication with the head second passageway 50 and the shaft passageway 32. The head second passageway 50 has a head second passageway inside diameter 53, a head second passageway lengthwise axis 55 that is centrally located through the head second passageway 50, a head second passageway 50 cross-sectional configuration, extends from the head first passageway 48 toward the head second end 40 to the head third passageway 52, and is in communication with the head first passageway 48 and the head third passageway 52. The head third passageway 52 has a head third passageway inside diameter 57, a head third passageway lengthwise axis 59 that is centrally located through the head third passageway 52, a head third passageway 52 cross-sectional configuration, extends from the head second passageway 50 to the head second end 40, and is in communication with the head second passageway 50 and an environment exterior to the head passageways 48, 50, 52.

The head first passageway inside diameter 49 is different than the head second passageway inside diameter 51. The head second passageway inside diameter 51 is different than the head third passageway inside diameter 53. In the illustrated embodiment, the head first passageway inside diameter 49 is equal to about the shaft passageway inside diameter 31, the head second passageway inside diameter 53 is greater than the shaft passageway inside diameter 31 and the head first passageway inside diameter 49, and the head second passageway inside diameter 51 is less than the head third passageway inside diameter 53. Each of the head passageways 48, 50, and 52 is sized and configured to receive a portion of the tissue manipulator 16, as described in more detail herein, and the head third passageway 52 is sized and configured to receive a portion of the set screw 14, as described in more detail herein. In the illustrated embodiment, the head first passageway lengthwise axis 51, the head second passageway lengthwise axis 55, and the head third passageway lengthwise axis 57 are coaxial.

The head first passageway 48 cross-sectional configuration is taken along a plane that is disposed orthogonally to the head first passageway lengthwise axis 51, the head second passageway 50 cross-sectional configuration is taken along a plane that is disposed orthogonally to the head second passageway lengthwise axis 55, and the head third passageway 52 cross-sectional configuration is taken along a plane that is disposed orthogonally to the head third passageway lengthwise axis 59. The head first passageway 48 cross-sectional configuration is different than the head second passageway 50 cross-sectional configuration, the head second passageway 50 cross-sectional configuration is different than the head third passageway 52 cross-sectional configuration, and the head third passageway 52 cross-sectional configuration is different than the head first passageway 48 cross-sectional configuration. In the illustrated embodiment, the head first passageway 48 has a circular cross-sectional configuration, the head second passageway 50 has a six-pointed star cross-sectional configuration that is sized and configured to receive a portion of a tool, such as a hand tool (e.g., star driver, delivery tool), and the head third passageway 52 has a circular cross-sectional configuration and defines the head thread 54.

The head thread 54 extends from the head inner surface 46 and into the head third passageway 52 toward the head third passageway lengthwise axis 59 and along a portion of the length of the head third passageway 52. In the illustrated embodiment, the head thread 54 extends from the head second end 40 toward the head first end 38. Each of the head first notch 56 and the head second notch 58 extends from the head second end 40 toward the head first end 38 and from the head outer surface 44 to the head inner surface 46 such that it is in communication with the head third passageway 52 and is sized and configured to receive a portion of the tissue manipulator 16. Each of the head first notch 56 and the head second notch 58 is disposed on an axis that is orthogonal to the head third passageway lengthwise axis 59 and has a cuboidal cross-sectional configuration.

An anchor can have any suitable dimensions according to an embodiment and selection of a suitable dimensions for an anchor can be based on various considerations, including the structural arrangement of the tissue intended to be treated using a tissue fixation system of which the anchor is a component. Examples of dimensions considered suitable for an anchor include those in which a head has a head outside diameter that is equal to, greater than, less than, or about 10 millimeters, a head has a head third passageway inside diameter that is equal to, greater than, less than, or about 8 millimeters, a head has a head second passageway that is sized and configured to receive a Torx T20 bit, a head has a head first passageway inside diameter that is equal to, greater than, less than, or about 1.2 millimeters, a head has a head first passageway inside diameter that is between about 0.254 millimeters and about 1.27 millimeters, a head has a head notch that has a width measured on a plane disposed orthogonal to a head third passageway lengthwise axis that is equal to, greater than, less than, or about 2 millimeters, a head has a head thread that is M8×0.5 threading, a head has a head length equal to, greater than, less than, or about 4 millimeters, a head has a head third passageway that has a length measured along the head third passageway lengthwise axis equal to, greater than, less than, or about 2 millimeters, a shaft has a shaft length equal to, greater than, less than, or about 5 millimeters, a shaft has a shaft outside diameter equal to, greater than, less than, or about 3 millimeters, a shaft has a shaft passageway inside diameter that is equal to, greater than, less than, or about 1.2 millimeters, a shaft has a shaft passageway inside diameter that is between about 0.254 millimeters and about 1.27 millimeters, a shaft has a shaft thread that is M3×0.5 threading with one or more flutes (e.g., two flutes), a set screw has a set screw outside diameter that is equal to, greater than, less than, or about 8 millimeters, a set screw has a set screw length equal to, greater than, less than, or about 2 millimeters, a set screw has a set screw thread that is M8×0.5 threading, a set screw has a set screw recess that is sized and configured to receive a Torx T20 bit, an anchor that utilizes metric threads, an anchor that utilizes English threads, and any other dimensions considered suitable for a particular embodiment.

While the head main body 42 has been illustrated as having a hollow circular cross-sectional configuration and taken along a plane that is disposed orthogonally to the head lengthwise axis 37, a head main body can have any suitable cross-sectional configuration that is taken along a plane that is disposed orthogonally to a head lengthwise axis. Selection of a suitable cross-sectional configuration for a head main body can be based on various considerations, including the intended use of an anchor of which the head is a component. Examples of cross-sectional configurations considered suitable for a head main body include hollow circles, hollow rectangles, hollow squares, hollow hexagons, and any other cross-sectional configuration considered suitable for a particular embodiment.

While the head first passageway 48 has been illustrated as having a circular cross-sectional configuration taken along a plane that is disposed orthogonally to the head first passageway lengthwise axis 51, the head second passageway 50 has been illustrated as having a six-pointed star cross-sectional configuration taken along a plane that is disposed orthogonally to the head second passageway lengthwise axis 55, and the head third passageway 52 has been illustrated as having a circular cross-sectional configuration taken along a plane that is disposed orthogonally to the head third passageway lengthwise axis 59, a head passageway can have any suitable cross-sectional configuration that is taken along a plane that is disposed orthogonally to a head passageway lengthwise axis. Selection of a suitable cross-sectional configuration for a head passageway can be based on various considerations, including the intended use of an anchor of which the head is a component. Examples of cross-sectional configurations considered suitable for a head passageway include those that are circular, rectangular, square, hexagonal, six-pointed stars, and any other cross-sectional configuration considered suitable for a particular embodiment.

While the head first passageway inside diameter 49 has been illustrated as being equal to about the shaft passageway inside diameter 31, the head second passageway inside diameter 53 has been illustrated as being greater than the shaft passageway inside diameter 31 and the head first passageway inside diameter 49, and the head second passageway inside diameter 51 has been illustrated as being less than the head third passageway inside diameter 53, a head passageway can have any suitable inside diameter. Selection of a suitable inside diameter for a head passageway can be based on various considerations, including the structural arrangement of a tissue manipulator intended to be disposed through the head and/or a set screw intended to be releasably attached to the head. Examples of diameters considered suitable for a head passageway include diameters that are greater than, less than, equal to, or about the diameter of a shaft passageway inside diameter, a head first passageway inside diameter, a head second passageway inside diameter, a head third passageway inside diameter, and any other diameter considered suitable for a particular embodiment.

While the head main body 42 has been illustrated as defining a head first passageway 48, a head second passageway 50, and head third passageway 52, a head main body can define any suitable number of passageways and selection of a suitable number of passageways for a head main body to define can be based on various considerations, including the structural arrangement of a set screw intended to interact with a head. Examples of numbers of passageways considered suitable for a head main body to define include one, at least one, two, a plurality, three, four, five, and any other number considered suitable for a particular embodiment.

While the head first passageway lengthwise axis 51, the head second passageway lengthwise axis 55, and the head third passageway lengthwise axis 57 have been illustrated as being coaxial, a head passageway lengthwise axis can be disposed at any angle relative to another head passageway lengthwise axis. Selection of a suitable angle to position a head passageway lengthwise axis relative to another head passageway lengthwise axis can be based on various considerations, including the structural arrangement of a tissue manipulator intended to be passed through a head and/or the structural arrangement of a set screw intended to be releasably attached to the head. Examples of angles considered suitable to position a head passageway lengthwise axis relative to another head passageway lengthwise axis include angles such that a first passageway lengthwise axis is disposed coaxially, parallel to, or at an obtuse, acute, or a right angle relative to a second passageway lengthwise axis, and any other angle considered suitable for a particular embodiment.

While the head thread 54 has been illustrated as extending along a portion of the length of the head third passageway 52, a head thread can have any suitable structural arrangement and extend along any suitable portion of a head. Selection of a suitable structural arrangement for a head thread and of a suitable portion of a head passageway length for a head thread to extend can be based on various considerations, including the material that forms a set screw intended to interact with the head thread. Examples of structural arrangements considered suitable for a head thread include those that are self-tapping, V threads, American National threads, unified threads, Whitworth threads, square threads, acme threads, buttress threads, knuckle threads, single start threads, double start threads, triple start threads, self-tapping threads, threads that have any suitable number of flutes, and any other structural arrangement considered suitable for a particular embodiment. Examples of portions of a head for a head thread to extend include along an entire head passageway length, along a portion of a head passageway length, from a head passageway first end to a head passageway second end, between a head passageway first end to a head passageway second end, from a head passageway first end toward a head passageway second end, from a head passageway second end toward a head passageway first end, along an entire head length, along a portion of a head length, from a head first end to a head second end, between a head first end to a head second end, from a head first end toward a head second end, from a head second end toward a head first end, and any other length considered suitable for a particular embodiment.

While the head main body 42 has been illustrated as defining a head first notch 56 and the head second notch 58 that are disposed on an axis that is orthogonal to the head third passageway lengthwise axis 59 and that have a cuboidal cross-sectional configuration, a head main body can define any suitable number of notches that have any suitable structural arrangement and are disposed on any suitable axis. Examples of numbers of notches considered suitable for a head main body to define include one, at least one, two, a plurality, three, four, five, and any other number considered suitable for a particular embodiment. Alternatively, a head main body can define a passageway that extends through the head main body and provides access to an environment exterior to a head passageway and the head passageway (e.g., head first passageway, head second passageway, head third passageway). Examples of structural arrangements considered suitable for a notch include cubes, cuboids, partial spheres, cylinders, prisms, structural arrangements that mirror the structural arrangement of a tissue manipulator (e.g., cross-sectional configuration of a tissue manipulator), and any other structural arrangement considered suitable for a particular embodiment. Examples of locations considered suitable to position a plurality of notches include positioning a first notch on a first axis that is disposed at any suitable angle relative to a head passageway lengthwise axis, such as a right angle, an obtuse angle, an acute angle, or at any other suitable angle relative to a second axis on which a second notch is disposed, such as a right angle, an obtuse angle, an acute angle, or any other angle considered suitable for a particular embodiment.

The set screw 14 is sized and configured to be releasably attached to the anchor 12 and partially disposed within the head third passageway 52 such that the set screw 14 releasably attaches the tissue manipulator 16 to the anchor 12. The set screw 14 has a set screw first end 60, a set screw second end 62, a set screw lengthwise axis 61, a set screw length 63 that extends from the set screw first end 60 to the set screw second end 62, a set screw outside diameter 65, and a set screw main body 64 that defines a set screw outer surface 66, a set screw recess 68, and a set screw thread 70. The set screw lengthwise axis 61 is centrally located through the set screw first end 60 and the set screw second end 62. The set screw outside diameter 65 is less than the head outside diameter 39 such that the set screw 14 is sized and configured to be received within the head third passageway 52. The set screw recess 68 extends from the set screw second end 62 toward the set screw first end 60 and has a first cross-sectional configuration. In the illustrated embodiment, the set screw recess 68 has a six-pointed star cross-sectional configuration that is taken along a plane that is disposed orthogonally to the set screw lengthwise axis 61 and is sized and configured to receive a portion of a tool, such as a hand tool (e.g., driver, delivery tool). The set screw recess 68 cross-sectional configuration is the same as the cross-sectional configuration of the head second passageway 50. The set screw thread 70 extends from the set screw outer surface 66 and along a portion of the set screw length 63 and is sized and configured to mate with the head thread 54.

While the set screw recess 68 has been illustrated as having a six-pointed star cross-sectional configuration taken along a plane that is disposed orthogonally to the set screw lengthwise axis 61, a set screw recess can have any suitable cross-sectional configuration that is taken along a plane that is disposed orthogonally to a set screw lengthwise axis. Selection of a suitable cross-sectional configuration for a set screw recess can be based on various considerations, including the intended use of an anchor of which the set screw is a component. Examples of cross-sectional configurations considered suitable for a set screw recess include those that are circular, rectangular, square, hexagonal, six-pointed stars, those that are the same as the cross-sectional configuration of a head passageway, and any other cross-sectional configuration considered suitable for a particular embodiment.

While the set screw thread 70 has been illustrated as extending along a portion of the set screw length 63, a set screw thread can have any suitable structural arrangement and extend along any suitable portion of a set screw length. Selection of a suitable structural arrangement for a set screw thread and of a suitable portion of a set screw length for a set screw thread to extend can be based on various considerations, including the structural arrangement of a head thread. Examples of structural arrangements considered suitable for a set screw thread include those that are self-tapping, V threads, American National threads, unified threads, Whitworth threads, square threads, acme threads, buttress threads, knuckle threads, single start threads, double start threads, triple start threads, self-tapping threads, threads that have any suitable number of flutes, threads that mirror a head thread, and any other structural arrangement considered suitable for a particular embodiment. Examples of portions of a set screw length for a set screw thread to extend include along an entire set screw length, along a portion of a set screw length, from a set screw first end to a set screw second end, between a set screw first end to a set screw second end, from a set screw first end toward a set screw second end, from a set screw second end toward a set screw first end, and any other length considered suitable for a particular embodiment.

The anchor 12 and set screw 14 can be formed of any suitable material and selection of a suitable material to form an anchor and a set screw according to a particular embodiment can be based on various considerations, including the material that forms a tissue manipulator intended to be used with an anchor and a set screw. Example materials considered suitable to form an anchor and a set screw include rigid materials, semi-rigid materials, implant grade materials, implant grade rigid materials, implant grade semi-rigid materials, biocompatible materials, materials that can be made biocompatible, metals such as stainless steel, titanium, metal alloys, cobalt chromium alloys, nickel-titanium alloy (e.g., Nitinol), thermoplastics, polymers, polyetheretherketone (PEEK), Pebax (Pebax is a registered trademark of Ato Chimie Corporation of Allee des Vosges, Courbevoie, France), nylon, polyethylene, high-density polyethylene (HDPE), high-performance polyethylene (HPPE), polyurethane, silicone, acrylonitrile butadiene styrene (ABS), polyoxymethylene (e.g., acetal), materials that are transparent, materials that are partially transparent, combinations of the materials described herein, and any other material considered suitable for a particular embodiment. An anchor and a set screw can be formed using any suitable technique or method of manufacture and selection of a suitable technique or method of manufacture to form an anchor or a set screw can be based on various considerations, including the material that forms a tissue manipulator intended to be used with an anchor and a set screw. Example methods of manufacturing an anchor and/or a set screw include those described herein, conventional machining techniques and methods of manufacture, 3D printing, injection molding, and any other technique or method of manufacture considered suitable for a particular embodiment.

A tissue fixation system can include any suitable tissue manipulator that is sized and configured to be received within an anchor according to an embodiment. Selection of a suitable tissue manipulator can be based on various considerations, including the tissue intended to be manipulated by the tissue manipulator and/or the material that forms an anchor through which a tissue manipulator is intended to be disposed. Examples of tissue manipulators that are considered suitable include those described in co-pending U.S. Provisional Application No. 62/489,552, filed Apr. 25, 2017, and entitled Medical Devices, Tissue Manipulators, Tissue Fixation Systems, and Associated Methods and Kits, which is hereby incorporated by reference in its entirety into this application.

An example tissue manipulator that can be included in tissue fixation system 10 is tissue manipulator 16. Tissue manipulator 16 is formed of a round wire member 74 and has a tissue manipulator first end 76, a tissue manipulator second end 78, a tissue manipulator length 79, a tissue manipulator shaft 80, and a tissue manipulator coil 82. The tissue manipulator length 79 extends from the tissue manipulator first end 76 to the tissue manipulator second end 78.

The tissue manipulator shaft 80 has a tissue manipulator shaft lengthwise axis 81, a tissue manipulator shaft first end 84, a tissue manipulator shaft second end 86, and a tissue manipulator shaft main body 88 that defines a predefined tissue manipulator shaft bend 90. The tissue manipulator shaft lengthwise axis 81 extends through the tissue manipulator shaft 80 from the tissue manipulator shaft bend 90 to the tissue manipulator shaft second end 86. The tissue manipulator shaft main body 88 extends from the tissue manipulator shaft first end 84 to the tissue manipulator shaft second end 86 and has a tissue manipulator shaft first main body portion 92, a tissue manipulator shaft second main body portion 94, and a tissue manipulator shaft outside diameter 89. The tissue manipulator shaft first main body portion 92 extends from the tissue manipulator shaft first end 84 to the tissue manipulator shaft bend 90 and has a tissue manipulator shaft first main body portion length 91. The tissue manipulator shaft second main body portion 94 extends from the tissue manipulator shaft bend 90 to the tissue manipulator shaft second end 86 and has a tissue manipulator shaft second main body portion length 93. In the illustrated embodiment, the tissue manipulator shaft first main body portion length 91 is less than the tissue manipulator shaft second main body portion length 93 and the tissue manipulator shaft outside diameter 89 is continuous along the tissue manipulator shaft main body 88.

The tissue manipulator shaft bend 90 defines an angle 95 between the tissue manipulator shaft first main body portion 92 and the tissue manipulator shaft second main body portion 94. In the illustrated embodiment, the angle 95 is equal to about 90 degrees. This structural arrangement provides a mechanism for creating a crank that can be used to assist with introducing the tissue manipulator 16 into tissue, as described in more detail herein. Alternatively, a tissue manipulator can omit defining a shaft bend and include a control handle that is moveable between a first configuration and a second configuration. In the first configuration, the control handle is releasably attached to the tissue manipulator along a portion of the tissue manipulator that extends from the tissue manipulator first end toward the tissue manipulator second end. In the second configuration, the control handle is free of, and not attached to, the tissue manipulator such that the tissue manipulator can be releasably attached to an anchor, as described in more detail herein. Examples of control handles considered suitable to use with a tissue manipulator are described in more detail herein. While tissue manipulator 16 has been illustrated as including a tissue manipulator shaft bend 90, a tissue manipulator can alternatively omit the inclusion of a shaft band such that the shaft is substantially linear and is sized and configured to be received within a handle, as described in more detail herein.

The tissue manipulator coil 82 is disposed between the tissue manipulator first end 76 and the tissue manipulator second end 78 and is attached to the tissue manipulator shaft 80 at the tissue manipulator shaft second end 86. The tissue manipulator coil 82 has a tissue manipulator coil lengthwise axis 97, a tissue manipulator coil first end 96, a tissue manipulator coil second end 98 that defines a sharp tip, and a tissue manipulator coil main body 100 that extends from the tissue manipulator coil first end 96 to the tissue manipulator coil second end 98. The tissue manipulator coil main body 100 has a tissue manipulator coil main body length 101, a tissue manipulator coil first main body outside diameter 103, a tissue manipulator coil second main body outside diameter 105, and defines a plurality of tissue manipulator coil turns 102 creating a helical coil 104. The tissue manipulator coil lengthwise axis 97 is coaxial with the tissue manipulator shaft lengthwise axis 81 and extends through the plurality of tissue manipulator coil turns 102. The tissue manipulator coil main body length 101 extends from the tissue manipulator coil first end 96 to the tissue manipulator coil second end 98 and is measured along the tissue manipulator coil lengthwise axis 97. In the illustrated embodiment, the plurality of tissue manipulator coil turns 102 comprises 4 complete coil turns that extend counterclockwise about the tissue manipulator coil lengthwise axis 97 creating the helical coil 104. In the illustrated embodiment, the plurality of tissue manipulator coil turns 102 has a maximum tissue manipulator coil turn outside diameter 107 that is equal to about three times the tissue manipulator shaft outside diameter 89. In the embodiment illustrated, the helical coil 104 is a 45 degree helical coil and defines a space 106 between adjacent turns of the coil 104. The tissue manipulator coil first main body outside diameter 103 is greater than the tissue manipulator coil second main body outside diameter 105 and the outside diameter of the tissue manipulator coil main body 100 tapers from the tissue manipulator coil first end 96 to the tissue manipulator coil second end 98. Alternatively, a tissue manipulator coil main body can have a constant outside diameter except for a pointed coil second end. Optionally, a marking indicia can be included on a tissue manipulator shaft that is disposed a distance from the second end of tissue manipulator that is equal to the coil length and the length of a shaft of a needle, or the sum of the length of a shaft of a needle and the length of a baseplate attached to a needle, such that the marking indicia can identify when the coil is completely disposed outside of a needle during treatment. A marking indicia can be included on a tissue manipulator as described in more detail herein.

When in use, as described in more detail herein, the set screw 14 is moveable between a first position, a second position, and a third position. In the first position the set screw 14 is free of, and not attached to, the anchor 12. In the second positon, the set screw 14 is releasably attached to the anchor 12 and is disposed a first distance from the head first end 38. In the third position the set screw 14 is disposed a second distance from the head first end 38 that is less than the first distance. When assembled, as shown in FIG. 1, the tissue manipulator 16 is partially disposed through the anchor 12 and is releasably attached to the anchor 12. The tissue manipulator 16 is removable from the anchor 12 (e.g., can be withdrawn from anchor by applying force on tissue manipulator 16 using a human hand or a hand tool being manipulated by a human hand with, or without, applying force to any other component of a tissue fixation system) when the set screw 14 is in the first position and releasably attached to the anchor 12 when the set screw 14 is in the second position and the third position (e.g., cannot be withdrawn from anchor 212 by applying force on tissue manipulator 216 using a human hand or a hand tool being manipulated by a human hand unless the set screw 214 is moved to the first position). In the illustrated embodiment, when the set screw 14 is in the second position and the third position, the tissue manipulator 16 is partially disposed within the shaft passageway 32, the head first passageway 48, the head second passageway 50, the head third passageway 52, and the head first notch 56. In the illustrated embodiment, when the set screw 14 is in the third position, the tissue manipulator 16 is partially disposed within the head first notch 56 and between the anchor 12 and the set screw first end 60 such that the tissue manipulator 16 is in contact with the anchor 12 and the set screw first end 60.

The tissue manipulator 16 can be formed of any suitable material and selection of a suitable material to form a tissue manipulator according to a particular embodiment can be based on various considerations, including the material that forms an anchor and/or a set screw intended to be used within the tissue manipulator. Example materials considered suitable to form tissue manipulator include rigid materials, semi-rigid materials, implant grade materials, implant grade rigid materials, implant grade semi-rigid materials, biocompatible materials, materials that can be made biocompatible, metals such as stainless steel, titanium, metal alloys, cobalt chromium alloys, nickel-titanium alloy (e.g., Nitinol), thermoplastics, polymers, polyetheretherketone (PEEK), Pebax, nylon, polyethylene, high-density polyethylene (HDPE), high-performance polyethylene (HPPE), polyurethane, silicone, acrylonitrile butadiene styrene (ABS), polyoxymethylene (e.g., acetal), combinations of the materials described herein, and any other material considered suitable for a particular application. In the illustrated embodiment, the tissue manipulator 16 is formed of an implant-grade metal alloy. Alternative embodiments, however, can include a tissue manipulator that is formed of a material that is the same as, or different than, a material that forms an anchor and/or a set screw according to an embodiment. For example, if a tissue manipulator is formed of a material that is relatively more flexible than the material that forms an anchor and/or a set screw according to an embodiment (e.g., the tissue manipulator is formed of a material that has a durometer hardness that is less than the durometer hardness of the material that forms an anchor and/or a set screw), the material that forms the tissue manipulator can flex, or deform, when the set screw contacts the tissue manipulator to releasably attach the tissue manipulator to the anchor. A tissue manipulator can be formed using any suitable technique or method of manufacture and selection of a suitable technique or method of manufacture to form a tissue manipulator can be based on various considerations, including the material that forms an anchor and/or a set screw intended to be used with the tissue manipulator. Examples of techniques and methods of manufacturing a tissue manipulator considered suitable include conventional machining techniques and methods of manufacture, 3D printing, injection molding, bending deformation, winding deformation, heat setting, and any other technique or method of manufacture considered suitable for a particular embodiment.

While the tissue fixation system 10 has been illustrated as including the tissue manipulator 16, a tissue fixation can optionally omit the inclusion of a tissue manipulator in embodiments in which a tissue manipulator is provided separately, or in which an anchor and a set screw of a tissue fixation system can provide treatment without use of a tissue manipulator. In these alternative embodiments, a tissue fixation system will comprise an anchor according to an embodiment and a set screw according to an embodiment.

While the tissue manipulator 16 has been illustrated as being partially disposed through each of the head first passageway 48, the head second passageway 50, and the head third passageway 52, a tissue manipulator can be partially disposed through any suitable portion of a head. Selection of a suitable portion of a head to position a tissue manipulator can be based on various considerations, including the structural arrangement of a head of an anchor. Examples of passageways considered suitable to partially dispose a tissue manipulator include a head first passageway, a head second passageway, a head third passageway, and/or any other passageway considered suitable for a particular embodiment.

While the tissue manipulator 16 has been illustrated as being partially disposed within the head first notch 56 when the set screw is in the second position and the third position, a tissue manipulator can be positioned in any suitable notch defined by an anchor during use. Selection of a suitable notch to position a set screw during use can be based on various considerations, including the structural arrangement of the portion of a body to which an anchor is intended to be attached. Examples of notches considered suitable to position a portion of a tissue manipulator include those in which a tissue manipulator is partially disposed within a first notch defined by an anchor, a tissue manipulator is partially disposed within a second notch defined by an anchor, and any other notch considered suitable for a particular embodiment.

Optionally, a tissue fixation system can include a cap that is sized and configured to be disposed over a head of an anchor the tissue fixation system. The cap can define a recess that is sized and configured to receive a portion, or the entirety, of a head length such that the head, and a tissue manipulator partially disposed within the head, can be shielded from elements, or features, exterior to a head passageway.

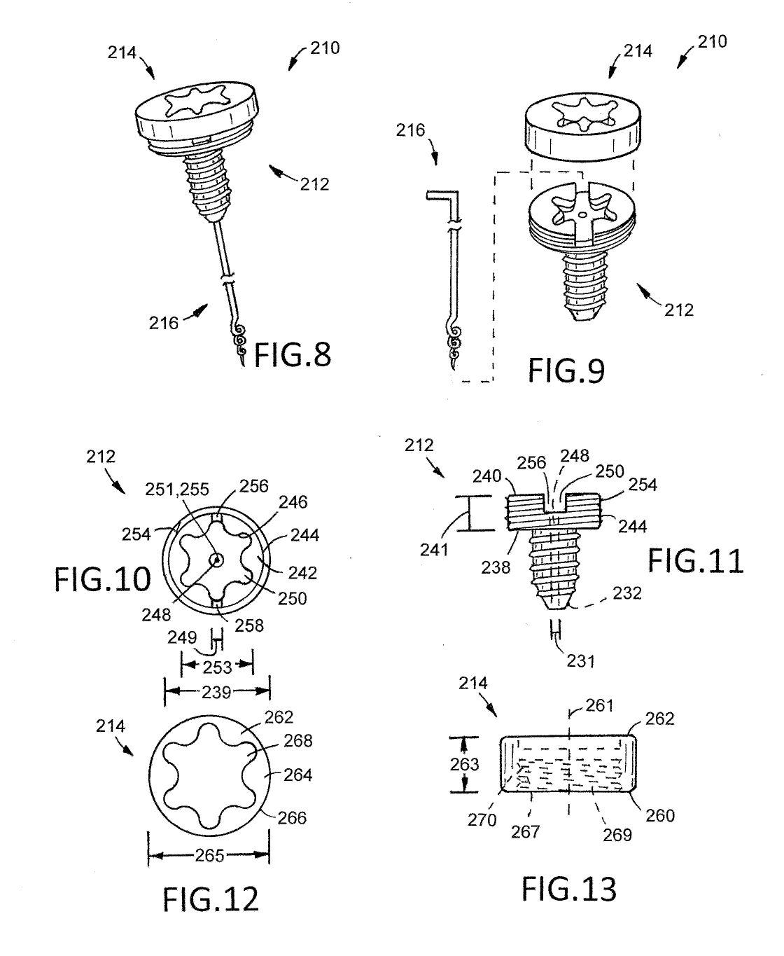

FIGS. 8, 9, 10, 11, 12, and 13 illustrate another example tissue fixation system 210. The tissue fixation system 210 is similar to the tissue fixation system 10 illustrated in FIGS. 1, 2, 3, 4, 5, 6, and 7 and described above, except as detailed below. The tissue fixation system 210 includes an anchor 212, a set screw 214, and a tissue manipulator 216. In the illustrated embodiment, the set screw 214 is releasably attached to the anchor 212 and the tissue manipulator 216 is partially disposed through, and releasably attached to, the anchor 212.

In the illustrated embodiment, the head main body 242 defines a head outer surface 244, a head inner surface 246, a head first passageway 248, a head second passageway 250, a head thread 254, a first head notch 256, and a second head notch 258.

The head first passageway 248 has a head first passageway inside diameter 249, a head first passageway lengthwise axis 251, a head first passageway 248 circular cross-sectional configuration, extends from the head first end 238 toward the head second end 240 to the head second passageway 250, and is in communication with the head second passageway 250 and the shaft passageway 232. The head second passageway 250 has a head second passageway inside diameter 253, a head second passageway lengthwise axis 255, a head second passageway 250 six-pointed star cross-sectional configuration that is sized and configured to receive a portion of a tool, such as a hand tool (e.g., delivery tool), extends from the head first passageway 248 to the head second end 240, and is in communication with the head first passageway 248 and an environment exterior to the head passageways 248, 250. The head first passageway inside diameter 249 is different than the head second passageway inside diameter 251. In the illustrated embodiment, the head first passageway inside diameter 249 is equal to about the shaft passageway inside diameter 231 and the head second passageway inside diameter 251 is greater than the head first passageway inside diameter 249. Each of the head passageways 248, 250 is sized and configured to receive a portion of the tissue manipulator 216. In the illustrated embodiment, the head first passageway lengthwise axis 251 and the head second passageway lengthwise axis 255 are coaxial.

The head thread 254 extends from the head outer surface 244 away from the head second passageway lengthwise axis 255 and along a portion of the head length 241. In the illustrated embodiment, the head thread 254 extends from the head second end 240 toward the head first end 238. Each of the first head notch 256 and the second head notch 258 extends from the head second end 240 toward the head first end 238 and from the head outer surface 244 to the head inner surface 246 such that it is in communication with the head second passageway 250 and is sized and configured to receive a portion of the tissue manipulator 216. Each of the first head notch 256 and the second head notch 258 is disposed on an axis that is orthogonal to the head second passageway lengthwise axis 255 and has a cuboidal cross-sectional configuration.

The set screw 214 is sized and configured to be releasably attached to the anchor 212 such that a portion of the anchor 212 is partially disposed within the set screw 214 and the tissue manipulator 216 is releasably attached to the anchor 212. The set screw 214 has a set screw first end 260, a set screw second end 262, a set screw lengthwise axis 261, a set screw length 263 that extends from the set screw first end 260 to the set screw second end 262, a set screw outside diameter 265, and a set screw main body 264 that defines a set screw outer surface 266, a set screw inner surface 267, a set screw first recess 268, a set screw second recess 269, and a set screw thread 270. The set screw lengthwise axis 261 is centrally located through the set screw first end 260 and the set screw second end 262. The set screw outside diameter 265 is greater than the head outside diameter 239 such that the head 222 is sized and configured to be receive within the set screw second recess 269. The set screw first recess 268 extends from the set screw second end 262 toward the set screw first end 260 and has a first cross-sectional configuration that is taken along a plane that is disposed orthogonally to the set screw lengthwise axis 261 and is the same as the cross-sectional configuration of the head second passageway 250. The set screw second recess 269 extends from the set screw first end 260 toward the set screw second end 262 and has a second cross-sectional configuration that is taken along a plane that is disposed orthogonally to the set screw lengthwise axis 261. The second cross-sectional configuration is different than the first cross-sectional configuration and is sized and configured to receive a portion of the anchor 212. The set screw thread 270 extends from the set screw inner surface 267, toward the set screw lengthwise axis 261, from the set screw first end 260 toward the set screw second end 262, along a portion of the set screw length 263 and is sized and configured to mate with the head thread 254.

When in use, the set screw 214 is moveable between a first position, a second position, and a third position. In the first position the set screw 214 is free of, and not attached to, the anchor 214. In the second position the set screw 214 is releasably attached to the anchor 214 and is disposed a first distance from the head first end 238. In the third position the set screw 214 is disposed a second distance from the head first end 238 that is less than the first distance. When assembled, as shown in FIG. 8, the tissue manipulator 216 is partially disposed through the anchor 212 and is releasably attached to the anchor 212. The tissue manipulator 216 is removable from the anchor 212 (e.g., can be withdrawn from anchor by applying force on tissue manipulator 216 using a human hand or a hand tool being manipulated by a human hand with, or without, applying force to any other component of a tissue fixation system) when the set screw 214 is in the first position and releasably attached to the anchor 212 when the set screw 214 is in the second position and the third position (e.g., cannot be withdrawn from anchor 212 by applying force on tissue manipulator 216 using a human hand or a hand tool being manipulated by a human hand unless the set screw 214 is moved to the first position). In the illustrated embodiment, when the set screw 214 is in the second position and the third position, the tissue manipulator 216 is partially disposed within the shaft passageway 232, the head first passageway 248, the head second passageway 250, and the head second notch 258. In the illustrated embodiment, when the set screw 214 is in the third position, the tissue manipulator 216 is partially disposed within the head second notch 258 and between the anchor 212 and the set screw first end 260 such that the tissue manipulator 216 is in contact with the anchor 212 and the set screw first end 260.

Examples of dimensions considered suitable for an anchor include those in which a head has a head outside diameter that is equal to, greater than, less than, or about 8 millimeters, a head has a head second passageway that is sized and configured to receive a Torx T20 bit, a head has a head first passageway inside diameter that is equal to, greater than, less than, or about 1.2 millimeters, a head has a head first passageway inside diameter that is between about 0.254 millimeters and about 1.27 millimeters, a head has a head notch that has a width measured on a plane disposed orthogonal to a head second passageway lengthwise axis that is equal to, greater than, less than, or about 2 millimeters, a head has a head thread that is M8×0.5 threading, a head has a head length equal to, greater than, less than, or about 4 millimeters, a head has a head second passageway that has a length measured along the head second passageway lengthwise axis equal to, greater than, less than, or about 2 millimeters, a shaft has a shaft length equal to, greater than, less than, or about 5 millimeters, a shaft has a shaft outside diameter equal to, greater than, less than, or about 3 millimeters, a shaft has a shaft passageway inside diameter that is equal to, greater than, less than, or about 1.2 millimeters, a shaft has a shaft passageway inside diameter that is between about 0.254 millimeters and about 1.27 millimeters, a shaft has a shaft thread that is M3×0.5 threading with two flutes, a set screw has a set screw outside diameter that is equal to, greater than, less than, or about 10 millimeters, a set screw has a set screw length equal to, greater than, less than, or about 4 millimeters, a set screw has a set screw thread that is M8×0.5 threading, a set screw has a set screw first recess that is sized and configured to receive a Torx T20 bit, a set screw has a set screw second recess that has a depth equal to, greater than, less than, or about 2 millimeters, a set screw has a set screw second recess that has a set screw second recess inside diameter equal to, greater than, less than, or about 8 millimeters, an anchor that utilizes metric threads, an anchor that utilizes English threads, and any other dimensions considered suitable for a particular embodiment.

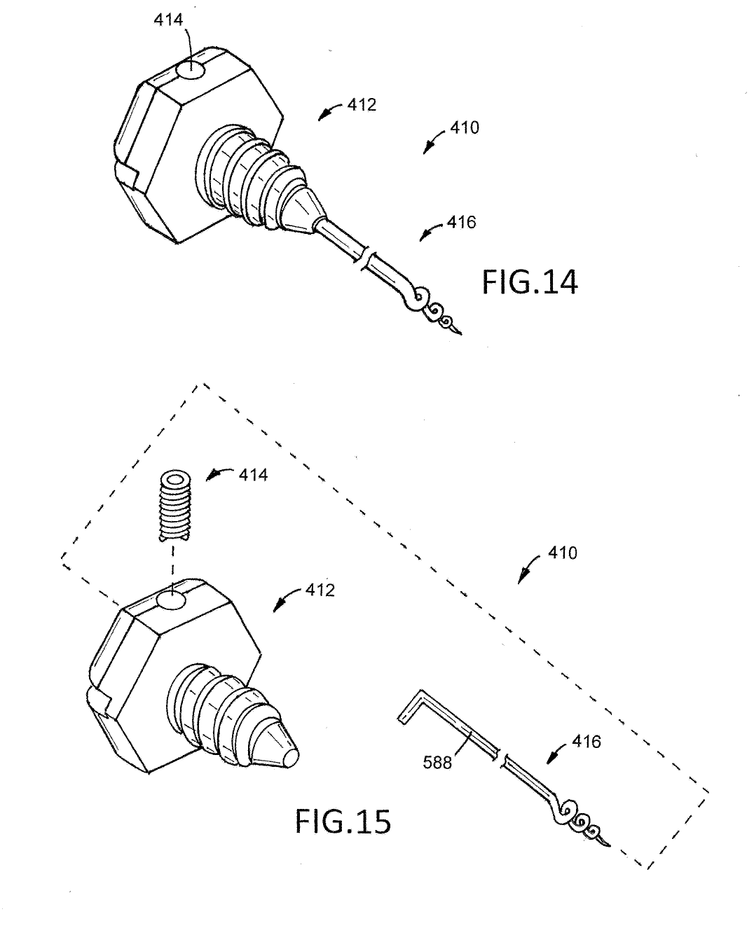

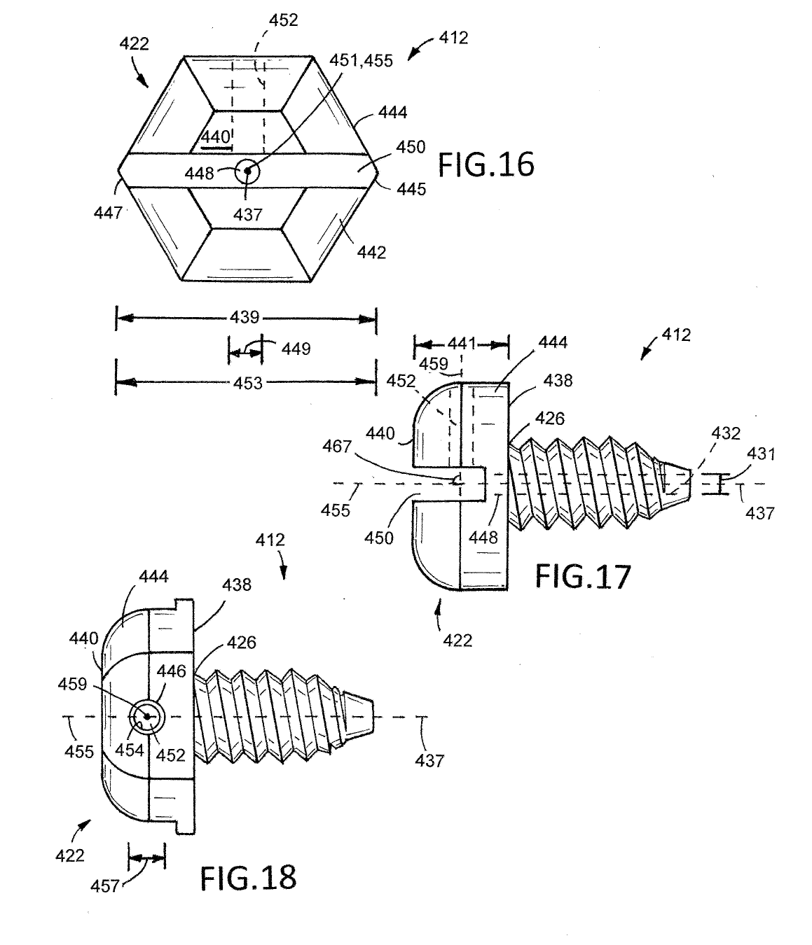

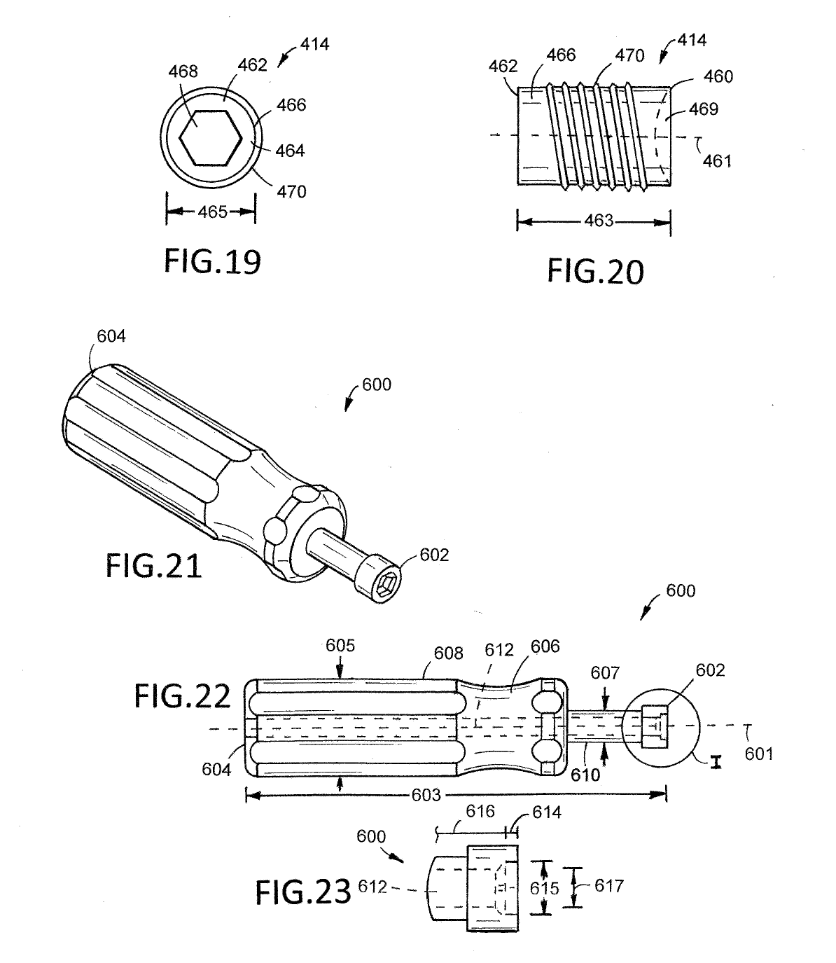

FIGS. 14, 15, 16, 17, 18, 19, and 20 illustrate another example tissue fixation system 410. The tissue fixation system 410 is similar to the tissue fixation system 10 illustrated in FIGS. 1, 2, 3, 4, 5, 6, and 7 and described above, except as detailed below. The tissue fixation system 410 includes an anchor 412, a set screw 414, and a tissue manipulator 416. In the illustrated embodiment, the set screw 414 is releasably attached to the anchor 412 and the tissue manipulator 416 is releasably attached to, and partially disposed through, the anchor 412.

The head 422 has a head first end 438 attached to the shaft second end 426, a head second end 440, a head lengthwise axis 437 that is centrally located through the head first end 438 and the head second end 440, a head outside diameter 439, a head length 441 that extends from the head first end 438 to the head second end 440, and a head main body 442 that defines a head outer surface 444, a head inner surface 446, a head first passageway 448, a head second passageway 450, a head third passageway 452, and a head thread 454. In the illustrated embodiment, the head main body 442 has a hexagonal cross-sectional configuration taken along a plane that is disposed orthogonally to the head lengthwise axis 437.

The head first passageway 448 has a head first passageway inside diameter 449, a head first passageway lengthwise axis 451 that is centrally located through the head first passageway 448, a head first passageway 448 cross-sectional configuration, extends from the head first end 438 toward the head second end 440 to the head second passageway 450, and is in communication with the shaft passageway 432 and the head second passageway 450. The head second passageway 450 has a head second passageway inside diameter 453, a head second passageway lengthwise axis 455 that is centrally located through the head second passageway 450, a head second passageway 450 cross-sectional configuration, extends from the head first passageway 448 to the head second end 440, and is in communication with the head first passageway 448 and an environment exterior to the head passageways 448, 450, 452. The head second passageway 450 is sized and configured to receive a portion of the tissue manipulator 416 and extends from a first side 445 of the head main body 442 to a second side 447 of the head main body 442 that is opposably facing the first side 445 such that the head second passageway 450 interrupts a portion of the head main body 442 that extends from the head second end 440 toward the head first end 438. The head third passageway 452 has a head third passageway inside diameter 457, a head third passageway lengthwise axis 459 that is centrally located through the head third passageway 452, a head third passageway 452 cross-sectional configuration, extends from the head second passageway 450 to the head outer surface 444, and is in communication with the head second passageway 450 and an environment exterior to the head passageways 448, 450, 452.

The head first passageway inside diameter 449 is different than the head second passageway inside diameter 453. The head second passageway inside diameter 453 is different than the head third passageway inside diameter 457. In the illustrated embodiment, the head first passageway inside diameter 449 is equal to about the shaft passageway inside diameter 431 and less than the head second passageway inside diameter 453 and the head second passageway inside diameter 453 is greater than the shaft passageway inside diameter 431, the head first passageway inside diameter 449, the head third passageway inside diameter 457, and is equal to about the head outside diameter 439. Each of the head first passageway 448 and head second passageway 450 is sized and configured to receive a portion of the tissue manipulator 416 and the head third passageway 452 is sized and configured to receive a portion of the set screw 414. In the illustrated embodiment, the head first passageway lengthwise axis 451 and the head second passageway lengthwise axis 455 are coaxial and the head third passageway lengthwise axis 459 is disposed at an angle 467 to the head second passageway lengthwise axis 455. In the illustrated embodiment, the angle 467 is equal to about 90 degrees.

The head first passageway 448 cross-sectional configuration is taken along a plane that is disposed orthogonally to the head first passageway lengthwise axis 451, the head second passageway 450 cross-sectional configuration is taken along a plane that is disposed orthogonally to the head second passageway lengthwise axis 455, and the head third passageway 452 cross-sectional configuration is taken along a plane that is disposed orthogonally to the head third passageway lengthwise axis 459. The head first passageway 448 cross-sectional configuration is different than the head second passageway 450 cross-sectional configuration, the head second passageway 450 cross-sectional configuration is different than the head third passageway 452 cross-sectional configuration, and the head third passageway 452 cross-sectional configuration is different than the head first passageway 448 cross-sectional configuration. In the illustrated embodiment, the head first passageway 448 has a circular cross-sectional configuration, the head second passageway 450 has a hexagonal cross-sectional configuration that is sized and configured to receive a portion of a tissue manipulator (e.g., shaft), as described in more detail herein, and/or a tool, such as a hand tool (e.g., flat head screw driver, delivery tool), and the head third passageway 452 has a circular cross-sectional configuration and defines the head thread 454. Alternative embodiments, however, can include a head second passageway that has a rectangular head second passageway cross-sectional configuration that does not extend to a head outer surface but does extend to a head second end. In this alternative embodiment, the head second passageway inside diameter is less than the head outside diameter. The head thread 454 extends from the head inner surface 446 into the head third passageway 452 toward the head third passageway lengthwise axis 459 and a long a portion of the length of the head third passageway 452. In the illustrated embodiment, the head thread 454 extends from the head outer surface 444 toward the head second passageway 450.

While the head third passageway lengthwise axis 459 has been illustrated as being disposed at an angle 467 to the head second passageway lengthwise axis 455 that is equal to about 90 degrees, a head third passageway lengthwise axis can be disposed at any suitable angle relative to a head second passageway lengthwise axis. Selection of a suitable angle to position a head third passageway lengthwise axis relative to a head second passageway lengthwise axis can be based on various considerations, including the structural arrangement of a set screw intended to be used with an anchor. Examples of angles considered suitable to position a head third passageway lengthwise axis relative to a head second passageway lengthwise axis include angles that are obtuse, acute, orthogonal, and any other angle considered suitable for a particular embodiment.

The set screw 414 is sized and configured to be releasably attached to the anchor 412 and is partially disposed within the anchor 412. In the illustrated embodiment, the set screw 414 is partially disposed within the head third passageway 452 such that the set screw 414 releasably attaches the tissue manipulator 416 to the anchor 412. The set screw 414 has a set screw first end 460, a set screw second end 462, a set screw lengthwise axis 461, a set screw length 463 that extends from the set screw first end 460 to the set screw second end 462, a set screw outside diameter 465, and a set screw main body 464 that defines a set screw outer surface 466, a set screw first recess 468, a set screw second recess 469, and a set screw thread 470. The set screw lengthwise axis 461 is centrally located through the set screw first end 460 and the set screw second end 462. The set screw outside diameter 465 is less than the head outside diameter 439 such that the set screw 414 is sized and configured to be received within the head third passageway 452. The set screw first recess 468 extends from the set screw second end 462 toward the set screw first end 460 and has a first cross-sectional configuration. In the illustrated embodiment, the set screw first recess 468 has a hexagonal cross-sectional configuration taken along a plane that is disposed orthogonally to the set screw lengthwise axis 461, is different than the cross-sectional configuration of the head second passageway 450, and is sized and configured to receive a portion of a tool, such as a hand tool (e.g., driver, delivery tool). The set screw second recess 469 extends from the set screw first end 460 toward the set screw second end 462 and is sized and configured to receive a portion of, or interact with, the tissue manipulator 414. The inclusion of a set screw second recess 469, as shown in FIG. 20, is considered advantageous at least because it provides a mechanism for pinching the tissue manipulator 416 between the set screw 414 and the anchor 412 within the head second passageway 450 to decrease, or eliminate, relative movement between the tissue manipulator 416 and anchor 412. For example, during use, the set screw first end 460 can become embedded within the tissue manipulator shaft main body 588 such that the tissue manipulator is pinched between the set screw first end 460 and the set screw head. The set screw thread 470 extends from the set screw outer surface 466, away from the set screw lengthwise axis 461, along a portion of the set screw length 463 and is sized and configured to mate with the head thread 454. A set screw included in a tissue fixation system can be partially disposed within a head passageway (e.g., head third passageway), entirely disposed within a head passageway, or provided separately from the head prior to use. It is considered advantageous to position a set screw such that it is partially disposed within a head passageway (e.g., head third passageway) prior to use such that a user need only advance the set screw into the head passageway to accomplish releasable attachment between the anchor, the set screw, and the tissue manipulator.

When in use, the set screw 414 is moveable between a first position, a second position, and a third position. In the first position the set screw 414 is free of, and not attached to, the anchor 412. In the second position the set screw 414 is releasably attached to the anchor 412 within the head third passageway 452 and is disposed a first distance from the head second passageway lengthwise axis 455. In the third position the set screw 414 is disposed within the head third passageway 452 and a second distance from the head second passageway lengthwise axis 455 that is less than the first distance. When assembled, as shown in FIG. 14, the tissue manipulator 416 is partially disposed through the anchor 412 and is releasably attached to the anchor 412. The tissue manipulator 416 is removable from the anchor 412 (e.g., can be withdrawn from anchor by applying force on tissue manipulator 416 using a human hand or a hand tool being manipulated by a human hand with, or without, applying force to any other component of a tissue fixation system) when the set screw 414 is in the first position and releasably attached to the anchor 412 when the set screw 414 is in the third position (e.g., cannot be withdrawn from anchor 412 by applying force on tissue manipulator 416 using a human hand or a hand tool being manipulated by a human hand unless the set screw 414 is moved to the first position or the second position). In the illustrated embodiment, when the set screw 414 is in the second position and the third position, the tissue manipulator 416 is partially disposed within the shaft passageway 432, the head first passageway 448, and the head second passageway 450. In the illustrated embodiment, when the set screw 414 is in the third position, the tissue manipulator 416 is partially disposed between the anchor 412 and the set screw first end 460 such that the tissue manipulator 416 is in contact with the anchor 412 and the set screw first end 460. It is considered advantageous to preload the set screw 414 within the head third passageway 452 (e.g., such that the set screw second end 462 is flush with head outer surface 444 such that a delivery tool can be used to attached anchor to tissue or bone) such that the set screw 414 can be advanced by a user without having to initially introduce the set screw 414 within the head third passageway 452.

Examples of dimensions considered suitable for an anchor include those in which a head has a head outside diameter that is equal to, greater than, less than, or about 6.35 millimeters, a head has a head length equal to, greater than, less than, or about 3 millimeters, a head has a head first passageway inside diameter that is equal to, greater than, less than, or about 1.2 millimeters, a head has a head first passageway inside diameter that is equal to, greater than, less than, or about 0.762 millimeters, a head has a head first passageway inside diameter that is between about 0.254 millimeters and about 1.27 millimeters, a head has a head second passageway that is sized and configured to receive a flat head screw driver, a head has a head second passageway that has a width measured on a plane disposed orthogonal to a head second passageway lengthwise axis that is equal to, greater than, less than, or about 1.016 millimeters, a head has a head second passageway that has a width measured on a plane disposed orthogonal to a head second passageway lengthwise axis that is between about 0.254 millimeters and about 1.27 millimeters, a head has a head second passageway that has a length measured along the head second passageway lengthwise axis that is equal to, greater than, less than, or about 2.311 millimeters, a head has a head second passageway that has a length measured along the head second passageway lengthwise axis that is between about 1.524 millimeters and about 2.54 millimeters, a head has a head third passageway inside diameter that is equal to, greater than, less than, or about 0.8 millimeters, a head has a head second passageway that is disposed about 1.5 millimeters from a head first end, a head has a head second passageway that is centrally located between adjacent facets of a hexagonal structure, a head has a head third passageway length measured along the head third passageway lengthwise axis that is equal to, greater than, less than, or about 3.175 millimeters, a head has a head thread that is M1.4×0.35 threading, a head defines a fillet that extends from the head second end toward the head first end and has a radius equal to, greater than, less than, or about 1.5 millimeters, a shaft has a shaft passageway inside diameter that is equal to, greater than, less than, or about 1.2 millimeters, a shaft has a shaft passageway inside diameter that is equal to, greater than, less than, or about 0.762 millimeters, a shaft has a shaft passageway inside diameter that is between about 0.254 millimeters and about 1.27 millimeters, a shaft has a plurality of flutes, a shaft that has 2 self-tapping flutes, a shaft that has 3 self-tapping flutes, a shaft has 4 self-tapping flutes, a shaft tapers from a location between the shaft first end and the shaft second end (e.g., between the shaft thread and the shaft first end) to the shaft first end, a shaft defines an angle between a surface that defines the shaft passageway and a tapered exterior surface of the shaft that extends from a location between the shaft first end and the shaft second end (e.g., between the shaft thread and the shaft first end) to the shaft first end that is equal to, greater than, less than, or about 45 degrees, a set screw has a set screw outside diameter that is between about 0.07 millimeters and about 2 millimeters, an anchor that utilizes metric threads, an anchor that utilizes English threads, a shaft has a shaft thread that is 8-36 threading with a plurality of flutes (e.g., four flutes), a shaft has a shaft thread that is M4×0.5 threading with a plurality of flutes (e.g., four flutes), a shaft has a shaft thread that corresponds to a #2, #3, #4, #5, or #6 sheet metal screw with one or more flutes, a set screw that is between about 0 and about 80 through about 2 and about 56 Imperial, between about M1.6×0.35 and about M2.5×0.45 Metric, a set screw has a set screw first recess diameter of about 0.7 mm, a set screw has a plan cup first end, a knurled cup first end, a flat first end, an oval first end, a coned first end, a half-dog first end, or a soft tipped first end, a shaft has a shaft length equal to, greater than, less than, or about 6.35 millimeters, and any other dimensions considered suitable for a particular embodiment. For example, a set screw that is 0-80 that has a 3/32 inch length with a 0.028 inch hex head is considered suitable.

In an alternative embodiment, an anchor can have a head that defines a fourth passageway that extends from a head outer surface to a head second passageway within which a head main body defines a second head thread sized and configured to mate with a second set screw. The fourth passageway of this alternative embodiment can be configured similar to the head third passageway 452, but positioned at a different location on the head, and the second set screw of this alternative embodiment can be configured similar to set screw 414.

FIGS. 21, 22, and 23 illustrate an example delivery tool 600 that can be used to implant a tissue fixation system, such as tissue fixation system 10, tissue fixation system 210, or tissue fixation system 410. The delivery tool 600 can be provided individually, or be included in a tissue fixation system, such as tissue fixation system 10, tissue fixation system 210, and/or tissue fixation system 410.

In the illustrated embodiment, the delivery tool 600 has a delivery tool first end 602, a delivery tool second end 604, a delivery tool lengthwise axis 601 that is centrally located through the delivery tool first end 602 and the delivery tool second end 604, a delivery tool length 603 that extends from the delivery tool first end 602 to the delivery tool second end 604, a delivery tool first outside diameter 605, a delivery tool second outside diameter 607, and a delivery tool main body 606 that defines a delivery tool handle 608, a delivery tool shaft 610, and a delivery tool passageway 612 that extends from the delivery tool first end 602 to the delivery tool second end 604. The delivery tool first outside diameter 605 is greater than the delivery tool second outside diameter 607.

The delivery tool passageway 608 has a delivery tool passageway first portion 614 and a delivery tool passageway second portion 616. The delivery tool passageway first portion 614 extends from the delivery tool first end 602 toward the delivery tool second end 604 to the delivery tool passageway second portion 616. The delivery tool passageway first portion 614 has a delivery tool passageway first portion inside diameter 615. The delivery tool passageway second portion 616 extends from the delivery tool passageway first portion 614 to the delivery tool second end 604. The delivery tool passageway second portion 616 has a delivery tool passageway second portion inside diameter 617. The delivery tool passageway first portion inside diameter 615 is greater than the delivery tool passageway second portion inside diameter 617. Each of the delivery tool passageway first portion inside diameter 615 and the delivery tool passageway second portion inside diameter 617 is sized and configured to receive a portion of a tissue manipulator and an anchor, such as those described herein.

In the illustrated embodiment, the delivery tool passageway first portion 614 has a first cross-sectional configuration and the delivery tool passageway second portion 616 has a second cross-sectional configuration that is different than the first cross-sectional configuration. The first cross-sectional configuration is hexagonal and is sized and configured to receive a portion of an anchor, such as anchor 412. The second cross-sectional configuration is circular. The delivery tool main body 606 defines a fillet between the delivery tool passageway first portion 614 and the delivery tool passageway second portion 616. This structural arrangement is considered advantageous at least because it provides a mechanism for passing an end of a tissue manipulator, such as tissue manipulator 16, into the delivery tool passageway 608 while preventing kinking and/or buckling of the tissue manipulator 16 as it is directed through the delivery tool passageway 608.

While the delivery tool 600 has been illustrated as having a particular structural arrangement, a delivery tool can have any suitable structural arrangement and selection of a suitable structural arrangement can be based on various considerations, such as the structural arrangement of a portion of a tissue fixation system, such as an anchor, a set screw, and/or a tissue manipulator that is intended be used with a delivery tool. Example structural arrangements considered suitable for a delivery tool include delivery tools that define a delivery tool passageway that has a constant inside diameter that extends from the delivery tool first end to the delivery tool second end, define a delivery tool passageway that has an inside diameter that varies from the delivery tool first end to the delivery tool second end, define a passageway that extends from a delivery tool first end toward a delivery tool second end, define a delivery tool passageway that extends along a portion, or the entirety, of a delivery tool length, and any other structural arrangement considered suitable for a particular embodiment.

While the delivery tool 600 has been illustrated as having a delivery tool passageway first portion 614 that is sized and configured to receive a portion of an anchor, such as anchor 412, a delivery tool can define any suitable structure capable of interacting with any suitable anchor according to an embodiment described herein, such as anchor 12, anchor 212, and/or anchor 412. Selection of a suitable structural arrangement for a delivery tool can be based on various considerations, including the intended use of an anchor with which the delivery tool is intended to be used. For example, an alternative structural arrangement includes a delivery tool that separately from, or in combination with, the arrangement shown in FIGS. 20 and 21, defines a delivery tool first end that has a cross-sectional configuration that is sized and configured to be received by a passageway defined by an anchor (e.g., delivery tool defines a projection that is sized and configured (e.g., six-pointed star) to be received by a passageway defined by an anchor).

A delivery tool can be formed of any suitable material and selection of a suitable material to form a delivery tool according to a particular embodiment can be based on various considerations, including the material that forms an anchor, a set screw, and/or a tissue manipulator intended to be used with a delivery tool. Example materials considered suitable to form a delivery tool include rigid materials, semi-rigid materials, biocompatible materials, materials that can be made biocompatible, metals such as stainless steel, titanium, metal alloys, cobalt chromium alloys, nickel-titanium alloy (e.g., Nitinol), thermoplastics, polymers, polyetheretherketone (PEEK), Pebax, nylon, polyethylene, high-density polyethylene (HDPE), high-performance polyethylene (HPPE), polyurethane, silicone, acrylonitrile butadiene styrene (ABS), polyoxymethylene (e.g., acetal), materials that have a round cross-sectional configuration, materials that have a rectangular, elongated, or square, cross-sectional configuration, materials that have a first cross-sectional configuration along a first portion of its length (e.g., round) and a second cross-sectional configuration along a second portion of its length (e.g., rectangular), combinations of the materials described herein, materials that are transparent, materials that are partially transparent, materials that are translucent, and any other material considered suitable for a particular embodiment. It is considered advantageous to form a delivery tool, or a portion of a delivery tool (e.g., handle, shaft) of a transparent material, or a material that is partially transparent, at least because it provides a mechanism for viewing any element disposed within a delivery tool passageway during use (e.g., a tissue manipulator). A delivery tool can be formed using any suitable technique or method of manufacture and selection of a suitable technique or method of manufacture to form a delivery tool can be based on various considerations, including the material that forms the delivery tool. Examples of techniques and methods of manufacturing a delivery tool include those described herein, conventional machining techniques and methods of manufacture, 3D printing, injection molding, and any other technique or method of manufacture considered suitable for a particular embodiment.

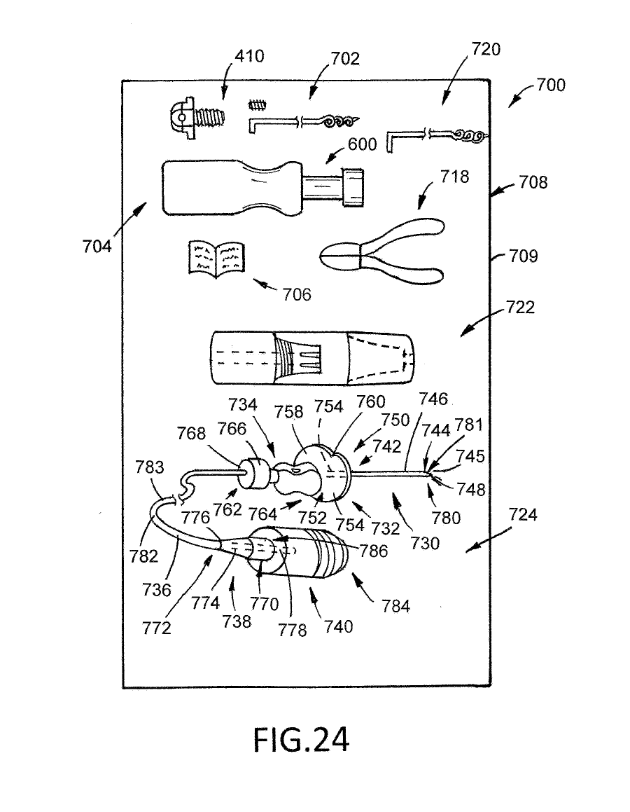

FIG. 24 illustrates an example embodiment of a kit 700 that includes a tissue fixation system 702 according to an embodiment; a delivery tool 704 according to an embodiment; instructions for use 706; and a storage container 708.

Any suitable tissue fixation system and delivery tool can be included in a kit and selection of a suitable tissue fixation system and delivery tool to include in a kit can be based on various considerations, including the tissue intended to be treated using the tissue fixation system. Examples of tissue fixation systems considered suitable to include in a kit include tissue fixation system 10, tissue fixation system 210, tissue fixation system 410, variations of the tissue fixation systems described herein, and any other tissue fixation system according to an embodiment. Examples of delivery tools considered suitable to include in a kit include delivery tool 600, variations of the delivery tools described herein, and any other delivery tool according to an embodiment. In the illustrated embodiment, the kit 700 includes tissue fixation system 410, as shown in FIGS. 14, 15, 16, 17, 18, 19, and 20 and delivery tool 600, as shown in FIGS. 21, 22, and 23. Optional additional components considered suitable to include in a kit are also illustrated in FIG. 24 and include snips 718; an additional tissue manipulator 720; a control handle 722; and a needle 724.

A kit can include any suitable control handle that is sized and configured to receive a portion of a tissue manipulator according to an embodiment and be releasably attached to a tissue manipulator according to an embodiment. Selection of a suitable control handle can be based on various considerations, including the tissue intended to be manipulated by the tissue manipulator and/or the material that forms a tissue manipulator. Examples of control handles that are considered suitable include those described in co-pending U.S. patent application Ser. No. 15/463,143, filed Mar. 20, 2017, and entitled Control Handles for Medical Devices, which is hereby incorporated by reference in its entirety into this application. The inclusion of a control handle in a kit is considered advantageous at least because it provides a mechanism for providing fine motor control over a tissue manipulator during use. A control handle can optionally be included in a tissue fixation system and be releasably attached to a tissue manipulator.

A kit can include any suitable needle that is sized and configured to receive a portion of a tissue manipulator according to an embodiment. Selection of a suitable needle can be based on various considerations, including the tissue intended to be manipulated by the tissue manipulator and/or the material that forms a tissue manipulator. An example of a needle considered suitable to include in a kit is illustrated in FIG. 24. The inclusion of needle 724 in a kit is considered advantageous at least because it provides a mechanism for providing transillumination during introduction of a tissue manipulator into tissue such that placement of the needle within the larynx, or other portion of the body, can be visualized (e.g., using a scope, such as a laryngoscope).

In the illustrated embodiment, the needle 724 comprises a shaft 730, a baseplate 732, a first connector 734, an optical fiber 736, a second connector 738, and a light source 740.

The shaft 730 has a first end 742, a second end 744, and a body 746 that defines a lumen 748 sized and configured to receive a portion of the optical fiber 736. In the illustrated embodiment, the first end 742 is attached to the baseplate 732 and the second end 744 defines a beveled sharp tip 745. A shaft included on a needle according to an embodiment can have any suitable structural arrangement and selection of a suitable structural arrangement for a shaft can be based on various considerations, including the treatment intended to be performed. Examples of structural arrangements for a shaft include those in which a shaft has a length that extends from the first end of the shaft to the second end of the shaft that is equal to, greater than, less than, or about 7 centimeters, shafts that are between about 10 gauge and about 25 gauge, between about 15 gauge and about 23 gauge, about 18 gauge, shafts that are ultra-thin, and any other structural arrangement considered suitable for a particular embodiment.

The baseplate 732 has a first end 750, a second end 752, and a body 754 that defines a lumen 756, a wall 758, and a notch 760. The first end 750 is attached to the first connector 734. The second end 752 is attached to the shaft 730. The lumen 756 is sized and configured to receive a portion of the optical fiber 736. The wall 758 extends outwardly from a lengthwise axis of the lumen 756 and provides structure for a user to manipulate the position of the needle 724 (e.g., shaft 730, second end 744) during use and have increased control over the needle 724 compared to needles that do not include a baseplate. The notch 760 extends from an outer surface of the wall 758 and toward the lengthwise axis of the lumen 756 and is disposed on a plane that extends from the lengthwise axis of the lumen 756 and contains the notch 760 and a portion of the beveled opening defined at the second end 744 of the shaft 730 such that the location of the beveled end of the shaft 730 can be determined during use. A baseplate included on a needle according to an embodiment can have any suitable structural arrangement and selection of a suitable structural arrangement for a baseplate can be based on various considerations, including the treatment intended to be performed. Examples of structural arrangements for a baseplate include those in which a shaft has a cross-sectional configuration that is circular, triangular, rectangular, square, pentagonal, hexagonal, and any other structural arrangement considered suitable for a particular embodiment. A baseplate can be attached to a shaft and a first connector using any suitable technique or method of attachment. Examples of suitable techniques and methods of attachment considered suitable between a baseplate and a shaft and between a baseplate and a first connector include using an adhesive, welding, fusing (e.g., heat fusing), threaded connections, integrated components, insert molding, and any other technique or method of attachment considered suitable for a particular application. Alternatively, a baseplate can be an integrated component of a shaft and/or a first connector.

The first connector 734 has a first end 762, a second end 764, and a body 766 that defines a lumen 768 sized and configured to receive a portion of the optical fiber 736. In the illustrated embodiment, the first end 762 is attached to the optical fiber 736 and the second end 764 is attached to the baseplate 732. The second end 764 is releasably attached to the baseplate 732 such that the first connector 734 and the optical fiber 736 can be removed from the needle 724 during use. The second connector 738 has a first end 770, a second end 772, and a body 774 that defines a lumen 776 sized and configured to receive a portion of the optical fiber 736. In the illustrated embodiment, the first end 762 is attached to the light source 740 and the second end 764 is attached to the optical fiber 736 (e.g., using heat shrink).

Each of the first connector 734 and second connector 738 can comprise any suitable type of connector and selection of a suitable type of connecter to include on a needle can be based on various considerations, including the type of treatment intended to be performed. Examples of connectors considered suitable to include on a needle include threaded connectors, Tuohy Borst adapters, luer lock connectors, male luer lock adaptors, MWCE male luer lock adaptors, Storz Connectors, and any other connector and/or adapter considered suitable for a particular embodiment. In the illustrated embodiment, the first connector 734 is a MWCE male luer lock adaptor and the second connector 738 is a Storz Connector.

The optical fiber 736 has a first end 778, a second end 780 that is releasably disposed within the lumen 748 defined by the shaft 730, and a body 782 that defines a light path 783 and is sized and configured to be received with the lumen 748 defined by the shaft 730, the lumen 756 defined by the baseplate 732, the lumen 768 defined by the first connector 734, and the lumen 776 defined by the second connector 738. In the illustrated embodiment, the first end 778 is operably connected to the light source 740 and the second end 780 defines a beveled tip 781 such that the beveled tip 745 of the shaft 730 and the beveled tip 781 of the optical fiber are disposed on a plane. This arrangement provides a mechanism for varying the light transmitted through tissue during use by rotating the needle and the beveled face of the optical fiber 736. Alternatively, a shaft and/or an optical fiber can omit the inclusion of a beveled end and define a rounded, squared, or other type of end. An optical fiber included on a needle according to an embodiment can have any suitable structural arrangement and selection of a suitable structural arrangement for an optical fiber can be based on various considerations, including the treatment intended to be performed. Examples of structural arrangements for an optical fiber include those in which an optical fiber has a length that extends from the first end of the optical fiber to the second end of the optical fiber that is between about 1 foot to about 10 feet, a length between about 2 feet and about 10 feet, a length greater than 10 feet, a length equal to about 4 feet, optical fibers that have an outside diameter about 0.040 inches, optical fibers that include an insulating jacket, optical fibers that have an outside diameter of about 0.087 inches when including an insulating jacket, optical fibers that have an outside diameter that varies along the length of the optical fiber, and any other structural arrangement considered suitable for a particular embodiment. The optical fiber 736 can comprise any suitable optical fiber. For example, commercially available optical fibers are considered suitable for use in the needles described herein and include, but are not limited to, plastic optical fibers and glass optical fibers, with or without cladding. Optionally, a second connector and an optical fiber can be wrapped in an insulating wrap.

The light source 740 has a first end 784 and a second end 786. The light source 740 is operatively connected to the optical fiber 736 such that light produced by the light source 740 can travel through the light path 783 defined by the optical fiber 736. The second end 786 is sized and configured to be attached to the second connector 738. The light source 740 can comprise any suitable light source and selection of a suitable light source can be based on various considerations, including the treatment intended to be completed. Example light sources considered suitable include in a needle include commercially-available light sources such as xenon, laser, LED, halogen light sources, and any other light source considered suitable for a particular embodiment. Optionally, light source 740 can include a fiber coupling (not shown) which provides communication between the light source and an optical fiber. A light source included on a needle according to an embodiment can have any suitable structural arrangement and selection of a suitable structural arrangement for a light source can be based on various considerations, including the treatment intended to be performed. A light source can be attached to an optical fiber and a second connector using any suitable technique or method of attachment. Examples of suitable techniques and methods of attachment considered suitable between a light source and an optical fiber and between a light source and a second connector include using an adhesive, welding, fusing (e.g., heat fusing), threaded connections, integrated components, insert molding, and any other technique or method of attachment considered suitable for a particular application. Alternatively, a light source can be an integrated component of a second connector.

A shaft, a baseplate, a first connector, an optical fiber, a second connector, and a light source can be formed of any suitable material and selection of a suitable material to form shaft, a baseplate, a first connector, an optical fiber, a second connector, and a light source can be based on various considerations, including the treatment intended to be accomplished. Examples of materials considered suitable to form a shaft, a baseplate, a first connector, an optical fiber, a second connector, and a light source include implant grade materials, biocompatible materials, materials that can be made biocompatible, metals such as stainless steel, aluminum, titanium, metal alloys, cobalt chromium alloys, nickel-titanium alloy (e.g., Nitinol), thermoplastics, polymers, polyetheretherketone (PEEK), Pebax, nylon, polyethylene, high-density polyethylene (HDPE), high-performance polyethylene (HPPE), polyurethane, silicone, acrylonitrile butadiene styrene (ABS), polyoxymethylene (e.g., acetal), materials that are transparent, materials that are partially transparent, combinations of the materials described herein, and any other material considered suitable for a particular embodiment. It is considered advantageous to form a second connector of nylon since this provides a mechanism for allowing a user to visualize the intensity of the light being transmitted through the optical fiber during use.

While the kit 700 has been illustrated as including a single tissue fixation system 702 and a single delivery tool 704, any suitable number, and type, of tissue fixation systems and/or delivery tools can be included in a kit. Selection of a suitable number of tissue fixation systems and/or delivery tools to include in a kit according to a particular embodiment can be based on various considerations, such as the treatment intended to be performed. Examples of suitable numbers of tissue fixation systems and/or delivery tools to include in a kit include at least one, one, two, a plurality, three, four and any other number considered suitable for a particular embodiment.

While the kit 700 has been illustrated as including optional components, such as snips 718, an additional tissue manipulator 720, a control handle 722, and a needle 724, a kit can include any suitable number of optional components, type of tool (e.g., snips), any suitable type of tissue manipulator, any suitable type of control handle, and any suitable needle. Examples of numbers of optional components considered suitable to include in a kit, such as tools, snips, tissue manipulators, control handles, and/or needles include one, at least one, two, a plurality, three, four, five, more than five, and any other number considered suitable for a particular embodiment. For example, an alternative embodiment of a kit can include a set screw delivery tool that is sized and configured to mate with and engage with a recess defined by the set screw such that it can be moved between its first, second, and third configurations.

A storage container included in a kit can have any suitable structural arrangement and be formed of any suitable material and selection of a suitable structural arrangement and material to form a storage container can be based on various considerations, including the number of tissue fixation systems and/or delivery tools included in a kit. Examples of structural arrangements considered suitable to form a storage container include boxes, boxes that include a lid, boxes that include a lid attached to the box (e.g., pivotably attached), bags, and any other structural arrangement considered suitable for a particular embodiment. Examples of materials considered suitable to form a storage container include metals, plastics, combinations of the materials described herein, and any other material considered suitable for a particular embodiment. In the illustrated embodiment, the storage container 708 is a box 709 formed of a rigid plastic.

In an alternative embodiment, a kit can include a first tissue fixation system, a second tissue fixation system, a first delivery tool, and a second delivery tool. The second tissue fixation system can be scaled relative to the first tissue fixation system. A kit that includes one or more tissue fixation systems that are scaled relative to one another provides a mechanism for selecting a tissue fixation system that best fits a particular patient's anatomy. For example, the first tissue fixation system can be sized and configured for a pediatric patient, the second tissue fixation system can be 2:1 scale of the first tissue fixation system and be sized and configured for teenage patient, or the second tissue fixation system can be 4:1 scale of the first tissue fixation system and be sized and configured for an adult patient. The first delivery tool can be sized and configured to interact with the first tissue fixation system. The second delivery tool can be sized and configured to interact with the second tissue fixation system. In embodiments in which more than one tissue fixation system is included in a kit, a second tissue fixation system can be scaled relative to a first tissue fixation system based on any suitable proportional ratio. Selection of a suitable amount to scale a second tissue fixation system relative to a first tissue fixation system can be based on various considerations, including the treatment intended to be performed. For example, a second tissue fixation system can be scaled such that it is 1:1, 2:1, 3:1, 4:1, 5:1, 6:1, 7:1 relative to a first tissue fixation system, between about 1:1 and about 7:1 relative to a first tissue fixation system, and any other proportional ratio considered suitable for a particular embodiment.

Various methods of treatment are described herein. While the methods described herein are shown and described as a series of acts, it is to be understood and appreciated that the methods are not limited by the order of acts, as some acts may in accordance with these methods, occur in different orders, and/or concurrently with other acts described herein.

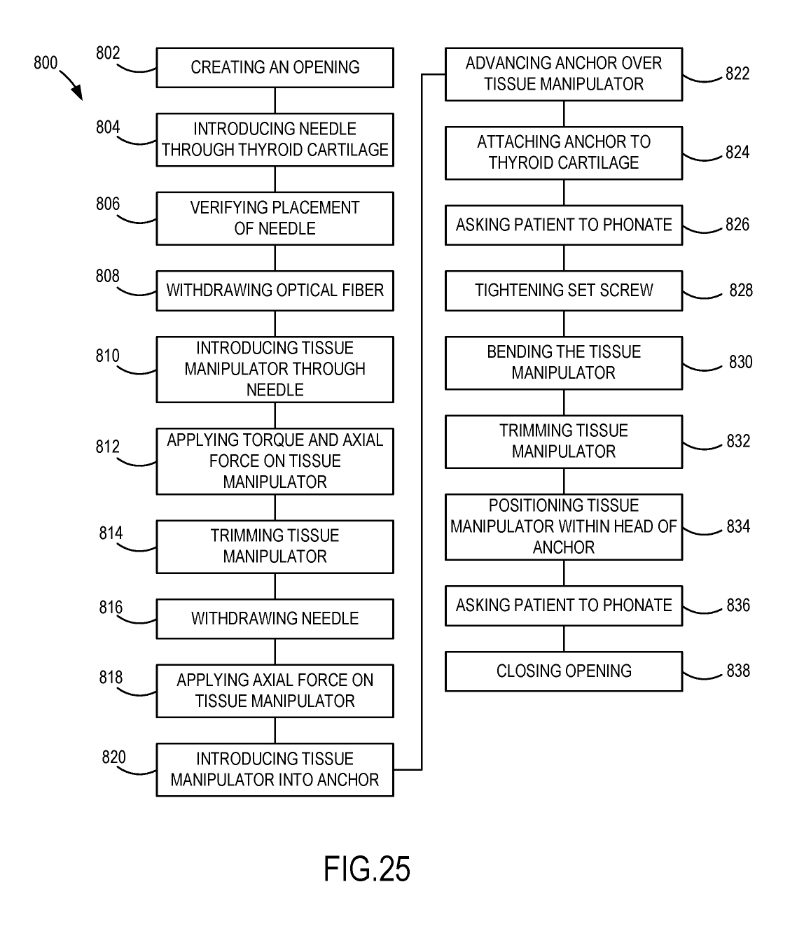

FIG. 25 is a schematic illustration of an example method of treatment 800 using a tissue fixation system and a delivery tool. In the example described herein, the method of treatment comprises a method of repositioning an arytenoid muscle resulting in repositioning of a vocal cord.

A step 802 comprises creating an opening in the body over the vocal cord intended to be treated to access the thyroid cartilage. Another step 804 comprises introducing a needle through the opening and the thyroid cartilage and into the arytenoid muscle. Another step 806 comprises verifying placement of the needle. Another step 808 comprises withdrawing the optical fiber from the needle while maintaining the position of the needle. Another step 810 comprises introducing a tissue manipulator through the needle until it contacts the arytenoid muscle. Another step 812 comprises applying torque on the tissue manipulator while applying an axial force toward the arytenoid muscle such that the tissue manipulator rotates and engages the arytenoid muscle. Another step 814 comprises trimming the tissue manipulator between the control handle and the shaft second end to create a new tissue manipulator end. Another step 816 comprises withdrawing the needle from the tissue and thyroid cartilage while maintaining position of the tissue manipulator. Another step 818 comprises applying an axial force on the tissue manipulator to achieve a desired placement of vocal cord. Another step 820 comprises introducing the new tissue manipulator end into a shaft passageway of an anchor. Another step 822 comprises advancing the anchor over the tissue manipulator until the anchor contacts the thyroid cartilage and such that a portion of the tissue manipulator is disposed within the anchor. Another step 824 comprises attaching the anchor to the thyroid cartilage. Another step 826 comprises asking the patient to phonate. Another step 828 comprises tightening the set screw onto the tissue manipulator while the portion of the tissue manipulator is disposed within a passageway of the head of the anchor. Another step 830 comprises bending a portion of the tissue manipulator that extends from the anchor relative to a portion that is disposed through the shaft passageway. Another step 832 comprises trimming the tissue manipulator. Another step 834 comprises positioning the portion of the tissue manipulator that has been bent relative to the portion that is disposed through the shaft passageway within a portion of the head of the anchor. Another step 836 comprises asking the patient to phonate to evaluate the position of the vocal cord and to determine whether adjustments are needed. Another step 838 comprises closing the opening made in the body.

Step 802 can be accomplished using any suitable medical device (e.g., scalpel, needle 724)) and by creating the opening on the side of the vocal cord that is being treated and over the thyroid cartilage. Step 802 can be accomplished by creating an opening that is sized and configured to allow a portion, or the entirety, of a tissue fixation system and/or delivery tool (e.g., tissue fixation system 10, tissue fixation system 210, tissue fixation system 410, delivery tool 600) to pass through the opening.

An optional step comprises administering medication near a point of treatment. This optional step can be accomplished prior to step 802 by administering any suitable medication at any suitable location on the body of a patient. For example, medication can be administered on the side of the vocal cord being treated and over the thyroid cartilage. Any suitable medication such as a local anesthetic, combination of local anesthetic and vasoconstrictor (e.g., 1% lidocaine, with 1:100,000 epinephrine), and/or general anesthetic can be used and administered in any suitable manner, such as subcutaneously using a convention syringe. Another optional step that can be completed prior to step 802 comprises asking the patient to phonate. This optional step can be accomplished by providing instructions to a patient to phonate. Another optional step that can be completed prior to step 802, or subsequent to step 802, comprises determining how a vocal cord needs to be modified for effective treatment based on patient's anatomy and/or phonation. This step can be accomplished by visualizing the vocal cord using a scope that is partially disposed in a patient's larynx and/or by listening to the patient phonate. Another optional step comprises dissecting the underlying soft tissues to expose the thyroid cartilage. This optional step can be accomplished subsequent to step 802 using any suitable medical device such that the thyroid cartilage is exposed. For example, this optional step can be accomplished using one or more of a needle, a probe, a pick, a scalpel, forceps, tweezers, scissors, or any other device considered suitable for a particular procedure.

Another optional step that can be completed subsequent to step 802 comprises creating one or more pilot holes through the thyroid cartilage sized and configured to receive a portion of a tissue fixation system and/or delivery tool (e.g., tissue fixation system 10, tissue fixation system 210, tissue fixation system 410, delivery tool 600). This optional step can be accomplished using any suitable drill and a drill bit that has a first outside diameter. A drill bit used complete any step of the methods described herein can have any suitable outside diameter, and skilled artisans will be able to select a suitable outside diameter for a drill bit according to a particular embodiment based on various considerations, including the structural arrangement at a point of treatment. Example outside diameters considered suitable for a drill bit include outside diameters that are equal to, less than, or greater than a shaft thread minor diameter. When a pilot hole is created, it is considered advantageous to use a drill bit with an outside diameter that is less than, or equal to, or about, a shaft thread minor diameter such that a friction fit can be accomplished between the anchor and the thyroid cartilage.

Step 804 can be accomplished using any needle considered suitable for a particular embodiment and selection of a suitable needle can be based on various considerations, such as the structural configuration of a tissue fixation system intended to be implanted. For example, needle 724 illustrated with respect to FIG. 24 is considered suitable to complete the methods described herein and is considered advantageous at least because it provides a mechanism for allowing identifying a desired treatment site using transillumination. Alternatively, step 804 can comprise introducing a needle through the opening and the thyroid cartilage and into the arytenoid muscle and omit the inclusion of an optical fiber. Alternatively, step 804 can comprise advancing the needle and obturator, or an obturator, through the opening, or through a pre-drilled pilot hole and into the arytenoid muscle.

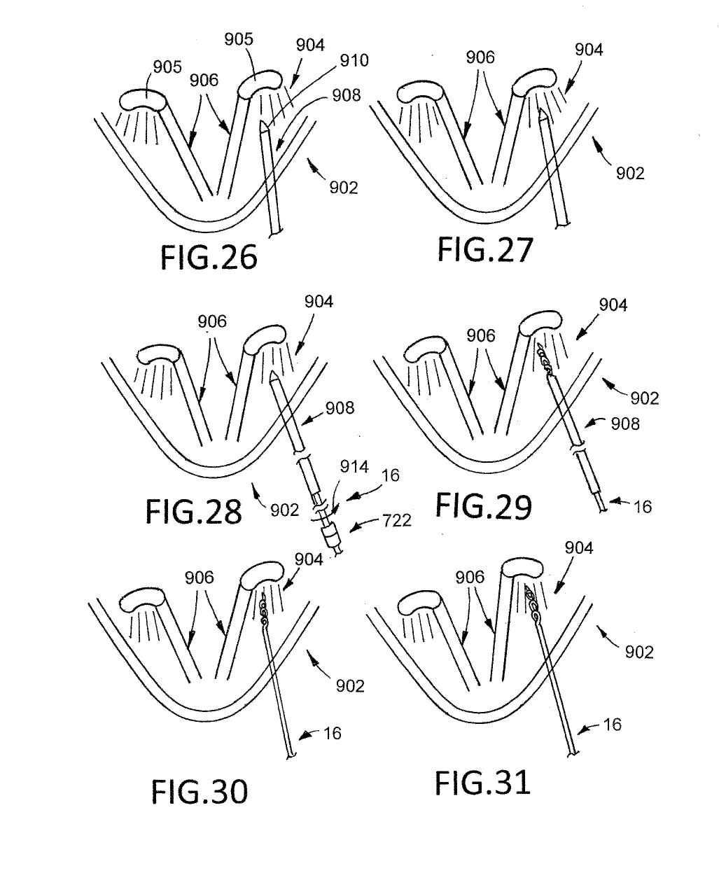

Step 804 can be accomplished by locating the thyroid cartilage, positioning the needle second end on the thyroid cartilage, and applying an axial force toward the thyroid cartilage on the needle such that it passes through the thyroid cartilage and into the arytenoid muscle (e.g., translaryngeal insertion of needle). It is considered advantageous to introduce the needle into the arytenoid muscle from the anterior to the posterior and to stop the application of the axial force on the needle before the needle second end penetrates the posterior surface of the arytenoid muscle. FIG. 26 illustrates a superior view of the thyroid cartilage 902, the arytenoid muscle 904, the arytenoid cartilage 905, and vocal cords 906 with the needle 908 and an optical fiber 910 disposed through the thyroid cartilage 902 and partially disposed within the arytenoid muscle 904.

Step 806 can be accomplished using a laryngoscope disposed within a portion of the larynx to facilitate direct visualization of the needle or facilitate transillumination to determine the location of the needle.

Step 808 can be accomplished by maintaining the position of the needle and applying an axial force on the optical fiber away from the thyroid cartilage until the optical fiber becomes free of the needle lumen. Alternatively, depending on the type of connector used as the first connector of the needle, torque may be applied to the first connector while maintain the position of the shaft of the needle to remove the optical fiber from the shaft. FIG. 27 illustrates the needle 908 disposed through the thyroid cartilage 902 and partially disposed within the arytenoid muscle 904 and the optical fiber 910 withdrawn from the needle lumen. In embodiments in which an optical fiber is not disposed within the needle lumen, step 808 can be omitted from method 800.

Step 810 can be accomplished by introducing the tissue manipulator second end into the needle lumen and applying an axial force on the tissue manipulator toward the thyroid cartilage until the tissue manipulator second end contacts the arytenoid muscle (e.g., inside the rearmost end of the arytenoid muscle). Step 810 can be accomplished by applying an axial force on any suitable portion (e.g., shaft) of any suitable tissue manipulator according to an embodiment. Selection of a suitable tissue manipulator to pass through a needle lumen can be based on various considerations, including the structural arrangement of the arytenoid muscle being treated. Example tissue manipulators considered suitable include tissue manipulator 16, tissue manipulator 216, tissue manipulator 416, variations of the tissue manipulators described herein, and any other tissue manipulator according to an embodiment. FIG. 28 illustrates a superior view of the thyroid cartilage 902 and the arytenoid muscle 904 with the needle 908 and tissue manipulator 16 disposed through the thyroid cartilage 902 and partially disposed within the arytenoid muscle 904. In the method of treatment 800, the tissue manipulator 16 illustrated and described with respect to FIGS. 1, 2, and 7 has been illustrated as being releasably attached to the arytenoid muscle 904 and an anchor 12. The tissue manipulator 16 includes a control handle 722 attached between the tissue manipulator first end and the tissue manipulator second end. Any suitable control handle can be used to accomplish the methods described herein and selection of a suitable control handle can be based on various considerations, such as the structural configuration of a tissue fixation system intended to be implanted. For example, control handle 722 illustrated with respect to FIG. 24 is considered suitable to complete the methods described herein and is considered advantageous at least because it provides a mechanism for providing tactile feedback during use and fine motor control over the tissue manipulator during use. Alternatively, step 810 can be accomplished without use of a control handle (e.g., in embodiment in which a tissue manipulator defines a predefined bend).

An optional step comprises obtaining a control handle. Another optional step comprises attaching the control handle to the tissue manipulator between the tissue manipulator first end and the coil. This step can be accomplished as described in co-pending U.S. patent application Ser. No. 15/463,143, filed Mar. 20, 2017, and entitled Control Handles for Medical Devices, which is hereby incorporated by reference in its entirety into this application.

Step 812 can be accomplished by applying torque on any suitable portion (e.g., shaft, first shaft main body portion, crank) of the tissue manipulator or the control handle while applying an axial force toward the arytenoid muscle such that the tissue manipulator rotates and engages the arytenoid muscle (e.g., sufficient purchase in the arytenoid is achieved). Depending on the direction in which the turns of the coil of the tissue manipulator are configured, torque can be applied on the tissue manipulator or control handle in either a clockwise or counterclockwise direction about the shaft lengthwise axis of the tissue manipulator. Arrow 914 illustrated in FIG. 28 shows the application of torque on the control handle 722 in a clockwise direction. This step is completed until approximately 3 or 4 turns of the tissue manipulator 16 are engaged with the tissue and such that the tissue manipulator 16 does not extend through the posterior surface of the arytenoid muscle. Alternatively, step 812 can comprise applying torque on the tissue manipulator or control handle such that the tissue manipulator rotates and engages the arytenoid muscle. This alternative step can be accomplished by applying torque on any suitable portion (e.g., shaft, first shaft main body portion, crank) of the tissue manipulator or control handle such that the tissue manipulator rotates and engages the arytenoid muscle (e.g., sufficient purchase in the arytenoid is achieved).

An optional step comprises confirming sufficient purchase of the tissue by the tissue manipulator. This optional step can be accomplished by directly visualizing the vocal cord, or via transillumination, using a scope that is partially disposed in a patient's larynx, listening to the patient phonate, and/or by tactile feedback from the tissue manipulator or control handle as the tissue manipulator is moved axially relative to the thyroid cartilage.

Step 814 can be accomplished using any suitable technique or method of trimming a tissue manipulator and selection of a suitable technique or method can be based on various considerations, such as the material(s) that forms the tissue manipulator. Examples of suitable techniques and methods of trimming a tissue manipulator include cutting using a cutting tool (e.g., snips), grinding, and any other suitable technique or method considered suitable for a particular embodiment. It is considered advantageous to trim the tissue manipulator between the control handle and the shaft second end to allow an anchor to be easily passed over the tissue manipulator, as described in more detail herein. Alternatively, in embodiments in which a control handle is not used, the tissue manipulator can be trimmed between the shaft bend and the shaft second end to allow an anchor to be easily passed over the tissue manipulator, as described in more detail herein. Alternative embodiments, however, can include a step that comprises trimming the tissue manipulator between the shaft bend and the shaft first end, trimming the tissue manipulator at the shaft bend, or omitting step 812. FIG. 29 illustrates the tissue manipulator 16 trimmed between the control handle and the shaft second end. Alternatively, step 812 can be accomplished by moving the control handle from the second configuration to the first configuration and removing the control handle from the tissue manipulator.

Step 816 can be accomplished by maintaining the position of the tissue manipulator and applying an axial force away from the thyroid cartilage on the needle until the needle is free of the thyroid cartilage. FIG. 30 illustrates the tissue manipulator 16 disposed through the thyroid cartilage 902 and partially disposed within the arytenoid muscle 904 and the needle withdrawn from the thyroid cartilage 902. Alternatively, step 814 and step 816 can be accomplished prior to step 812.

Step 818 can be accomplished by applying an axial force toward (e.g., adduction), or away from (e.g., abduction), the thyroid cartilage on any suitable portion of the shaft of the tissue manipulator to position the arytenoid muscle in a desired location that can be based on the patient's anatomy or that results in a desired phonation. Alternatively, torque can be applied separately, or in combination with the axial force, to the shaft of the tissue manipulator (e.g., clockwise or counterclockwise direction about the shaft lengthwise axis) to achieved desired placement of vocal cord. FIG. 31 illustrates the tissue manipulator 16 disposed through the thyroid cartilage 902 and partially disposed within the arytenoid muscle 904. The arytenoid muscle 904 has been positioned in a desired location such that the vocal cord 906 achieves the desired phonation.

Step 820 can be accomplished by positioning the shaft first end of an anchor toward the thyroid cartilage and applying an axial force on the anchor toward the thyroid cartilage such that the new tissue manipulator end passes into the shaft passageway. Step 820 can be accomplished by applying an axial force on any suitable portion (e.g., head of anchor) of the anchor. Any suitable anchor according to an embodiment can be used to complete the methods described herein. Selection of a suitable anchor can be based on various considerations, including the structural arrangement of the thyroid cartilage to which an anchor is intended to be attached. Examples of anchors and set screws considered suitable to complete the methods described herein include anchor 12, anchor 212, anchor 412, variations of the anchors described herein, set screw 14, set screw 214, set screw 414, variations of the set screws described herein, and any other anchor and/or set according to an embodiment. In the method of treatment 800, the anchor 412 and set screw 416 illustrated and described with respect to FIGS. 14, 15, 16, 17, 18, 19, and 20 have been illustrated as being releasably attached to the thyroid cartilage 902 and tissue manipulator 16. An optional step that can be included in method 800 comprises obtaining a tissue fixation system, such as a tissue fixation system according to an embodiment.

Step 822 can be accomplished by applying a force on any suitable portion of the anchor directed toward the thyroid cartilage until the anchor is advanced over the tissue manipulator, a portion of the tissue manipulator is disposed within the anchor, and the anchor contacts the thyroid cartilage.

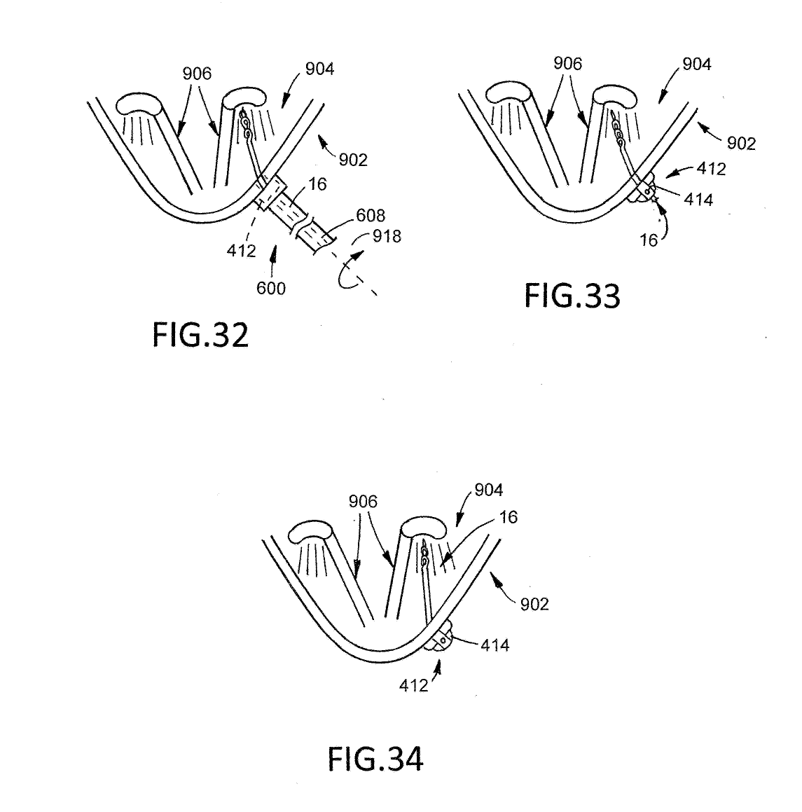

Step 824 can be accomplished by positioning a delivery tool over the head of the anchor and applying torque on the delivery tool such that the head of the anchor rotates relative to the thyroid cartilage. This step can be completed until the head first end is disposed adjacent to the thyroid cartilage. Any suitable delivery tool, including those according to an embodiment, can be used to complete the methods described herein. Selection of a suitable delivery tool can be based on various considerations, including the structural arrangement of the anchor intended to interact with the delivery tool. Examples of delivery tools considered suitable to complete the methods described herein delivery tool 600, variations of the delivery tools described herein, and any other delivery tool considered suitable for a particular embodiment. In the method of treatment 800, the delivery tool 600 illustrated and described with respect to FIGS. 21, 22, and 23 has been illustrated as being used to attach the anchor 412 to the thyroid cartilage. FIG. 32 illustrates the anchor 412 being attached to the thyroid cartilage 902 using delivery tool 600. In addition, FIG. 32 illustrates arrow 918 as the application of torque on the delivery tool 600 in a clockwise direction such that anchor 412 is advanced into the thyroid cartilage 902. It is considered advantageous to utilize delivery tool 600 to attach an anchor to the thyroid cartilage at least because the delivery tool 600 defines a delivery tool passageway 608 that is sized and configured to receive a portion of the tissue manipulator 16. As shown in FIG. 32, a portion of the tissue manipulator 16 is disposed within delivery tool passageway 608. Alternatively, in embodiments in which anchor 12 and/or anchor 212 are being attached to the thyroid cartilage, step 826 can be accomplished by positioning a delivery tool within a passageway defined by the head of the anchor (e.g., head third passageway 52 if anchor 12 is being used, head second passageway 250 if anchor 212 is being used) and applying torque on the delivery tool such that the head of the anchor rotates relative to the thyroid cartilage.

Step 826 can be accomplished by providing instructions to a patient to phonate to determine whether placement of the vocal cord is satisfactory.

Step 828 can be accomplished by applying torque on the set screw using any suitable tool, such as a driver, in a direction that results in the set screw advancing toward the tissue manipulator until the set screw contacts and releasably attaches the tissue manipulator to the anchor and the thyroid cartilage. This step can be continued until the set screw second end is disposed within the head third passageway and does not protrude from the head. This step advantageously releasably attaches the tissue manipulator between the anchor and the set screw. FIG. 33 illustrates the tissue manipulator 416 releasably attached to the anchor 412 and the thyroid cartilage 902 using the set screw 914. This configuration prevents the tissue manipulator from springing back toward its original configuration and provides a pinching point between the anchor and the set screw that pinches the tissue manipulator in place.

An optional step that can be completed subsequent to step 828 and subsequent to and/or concurrent with step 826 comprises adjusting the position of the tissue manipulator. This optional step can be accomplished by applying an axial force toward, or away from, the thyroid cartilage on any suitable portion of the shaft of the tissue manipulator to position the arytenoid muscle in a desired location that can be based on the patient's anatomy or that results in a desired phonation. Alternatively, torque can be applied separately, or in combination with the axial force, to the shaft of the tissue manipulator (e.g., clockwise or counterclockwise direction about the shaft lengthwise axis) to achieved desired placement of vocal cord. Another optional step comprises maintaining the position of the tissue manipulator in the desired location while step 828 is completed.

Step 830 can be accomplished by applying an axial force on the shaft of the tissue manipulator orthogonal to the shaft lengthwise axis and toward the midline of the throat, or in any other direction, such that a bend is formed along the shaft between the new tissue manipulator end and the shaft second end. FIG. 33 illustrates the tissue manipulator 16 bent toward the midline of the throat. In embodiments in which anchor 12 and/or anchor 212 are being used to complete method 800, step 830 can be accomplished prior to step 828 such that a portion of the tissue manipulator can be positioned within a notch prior to tightening the set screw. Alternatively, step 830 can be omitted from method 800 and step 832 can be accomplished.

Step 832 can be accomplished as described with respect to step 814. FIG. 33 illustrates a trimmed tissue manipulator 16 disposed through the anchor 12. For example, it is considered advantageous to trim the tissue manipulator such that a newly created tissue manipulator first end is disposed from the anchor 412 a distance that is less than the distance from the head first passageway and the head outer surface. Alternatively, step 832 can be accomplished prior to step 830. In embodiments in which anchor 12 and/or anchor 212 are being used to complete method 800, step 832 can be accomplished prior to step 828 or step 830 such that a portion of the tissue manipulator can be positioned within a notch prior to tightening the set screw. Alternatively, step 832 can be accomplished prior to step 830, which can be omitted from method 300, and such that the newly created tissue manipulator end is disposed within a passageway defined by the head of the anchor.

Step 834 can be accomplished by applying an axial force on the tissue manipulator directed toward the thyroid cartilage until the newly created tissue manipulator first end is disposed within a portion of the head. In the illustrated embodiment, step 834 can be accomplished by applying an axial force on the tissue manipulator directed toward the thyroid cartilage until the newly created tissue manipulator first end is disposed within the head second passageway. FIG. 34 illustrates the newly created tissue manipulator first end disposed within the head second passageway. Alternatively, in embodiments in which anchor 12 or anchor 212 is being used to attached the tissue manipulator to the thyroid cartilage, step 834 can be omitted from method 800.

Step 836 can be accomplished by providing instructions to a patient to phonate.

An optional step that can be completed subsequent to and/or concurrent with step 836 comprises adjusting the position of the tissue manipulator. This optional step can be accomplished by applying moving the set screw to the first or second position and applying an axial force toward, or away from, the thyroid cartilage on any suitable portion of the shaft of the tissue manipulator to position the arytenoid muscle in a desired location that can be based on the patient's anatomy or that results in a desired phonation. Alternatively, torque can be applied separately, or in combination with the axial force, to the shaft of the tissue manipulator (e.g., clockwise or counterclockwise direction about the shaft lengthwise axis) to achieved desired placement of vocal cord. Another optional step comprises maintaining the position of the tissue manipulator in the desired location while this optional step is completed. Another optional step comprises moving the set screw to the third position.

Step 838 can be accomplished using any suitable device and/or method, such as by suturing the opening created in step 802.

FIG. 34 illustrates the tissue fixation system 410 illustrated in FIGS. 14, 15, 16, 17, 18, 19, and 20 attached to the thyroid cartilage 902 and the tissue manipulator 16 disposed through the anchor 412 and the thyroid cartilage 902.

While method 800 has been described with respect to manipulating the position of an arytenoid muscle, any of the steps, alternative steps, and/or optional steps described herein can be utilized to treat any suitable tissue and a tissue fixation system can be attached to any suitable wall or feature of a patient. Selection of a suitable point of treatment to implant a tissue fixation system according to an embodiment can be based on various considerations, including the treatment intended to be performed and the structural arrangement at the treatment site.

While various steps, alternative steps, and optional steps have been described above with respect to the example method of treatment 800 using a tissue fixation system and a delivery tool, these steps, alternative steps, and optional steps can be included in, accomplished concurrently with, and/or accomplished in the alternative to, the method, steps, alternative steps, and/or optional steps described below with respect to the example method of treatment 1000.

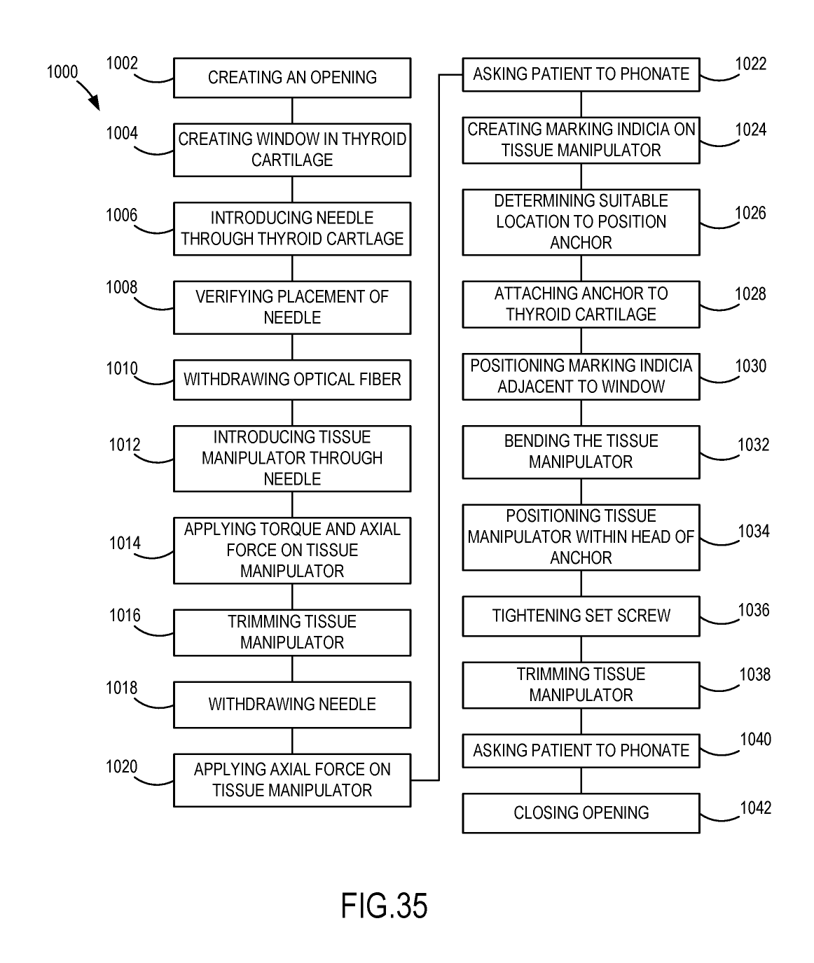

FIG. 35 is a schematic illustration of an example method of treatment 1000 using a tissue fixation system and a delivery tool. In the example described herein, the method of treatment comprises a method of repositioning an arytenoid muscle resulting in repositioning of a vocal cord.

A step 1002 comprises creating an opening in the body over the vocal cord intended to be treated to access the thyroid cartilage. Another step 1004 comprises creating a window in the thyroid cartilage. Another step 1006 comprises introducing a needle through the window and into the arytenoid muscle. Another step 1008 comprises verifying placement of the needle. Another step 1010 comprises withdrawing the optical fiber while maintaining the position of the needle. Another step 1012 comprises introducing a tissue manipulator through the needle until it contacts the arytenoid muscle. Another step 1014 comprises applying torque on the tissue manipulator while applying an axial force toward the arytenoid muscle such that the tissue manipulator rotates and engages the arytenoid muscle. Another step 1016 comprises trimming the tissue manipulator between the control handle and the shaft second end to create a new tissue manipulator end. Another step 1018 comprises withdrawing the needle from the tissue and thyroid cartilage while maintaining position of the tissue manipulator. Another step 1020 comprises applying an axial force on the tissue manipulator to achieve a desired placement of vocal cord. Another step 1022 comprises asking the patient to phonate. Another step 1024 comprises creating a marking indicia on the shaft of the tissue manipulator where it exits the thyroid cartilage while holding the tissue manipulator at desired location. Another step 1026 comprises determining a suitable location to position an anchor relative to the window created in the thyroid cartilage. Another step 1028 comprises attaching the anchor to the thyroid cartilage. Another step 1030 comprises positioning the marking indicia on the tissue manipulator adjacent to the window. Another step 1032 comprises bending a portion of the tissue manipulator that extends from the window at the mark and relative to a portion that extends through the thyroid cartilage. Another step 1034 positioning the bent portion of the tissue manipulator within a portion of the anchor. Another step 1036 tightening the set screw to the tissue manipulator while the portion of the tissue manipulator is disposed within a portion of the anchor. Another step 1038 comprises trimming the tissue manipulator. Another step 1040 comprises asking the patient to phonate to evaluate the position of the vocal cord and to determine whether adjustments are needed. Another step 1042 comprises closing the opening made in the body.

Step 1002 can be accomplished as described herein with respect to step 802.

Step 1004 can be accomplished using any suitable medical device (e.g., scalpel, needle) and by creating the window on the side of the vocal cord that is being treated and through the thyroid cartilage. As used herein, the term “window” refers to an opening through a wall of a portion of the body, such as the wall of the thyroid cartilage. Step 1004 can be accomplished by creating a window that is sized and configured to allow a portion of a tissue fixation system, such as a tissue manipulator, to pass through the window.

Step 1006 can be accomplished as described herein with respect to step 804. Step 1008 can be accomplished as described herein with respect to step 806. Step 1010 can be accomplished as described herein with respect to step 808. Step 1012 can be accomplished as described herein with respect to step 810. Step 1014 can be accomplished as described herein with respect to step 812. Step 1016 can be accomplished as described herein with respect to step 814. Step 1018 can be accomplished as described herein with respect to step 816. Step 1020 can be accomplished as described herein with respect to step 818. Step 1022 can be accomplished as described herein with respect to step 826.

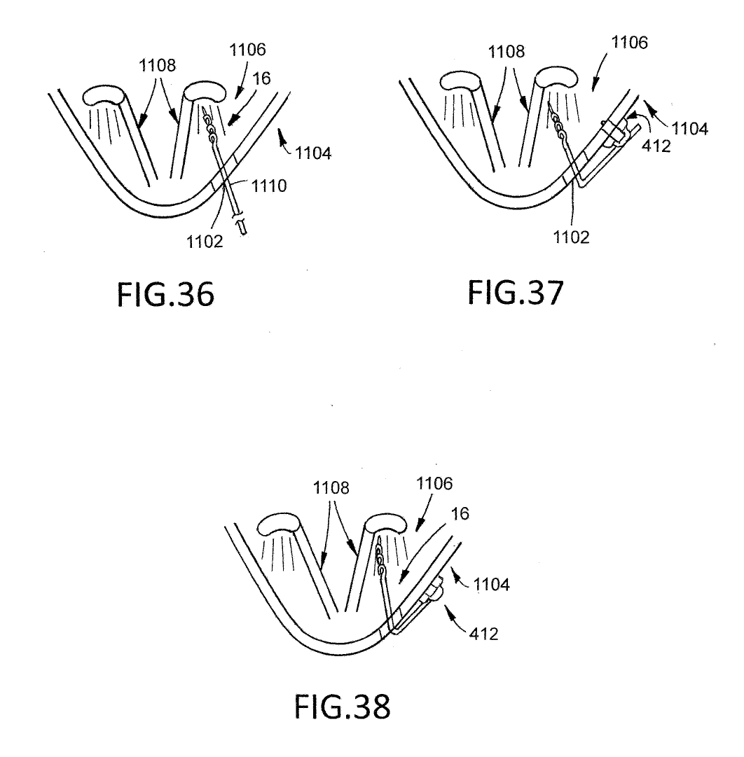

Step 1024 can be accomplished using any suitable marking implement such as writing implements, pens, markers, and any other marking implement considered suitable for a particular embodiment. Step 1024 can be accomplished while holding the tissue manipulator in a desired location in which a desired phonation can be produced. Alternatively, step 1024 can comprise creating a marking indicia on the shaft of the tissue manipulator at a location that is disposed a distance from the thyroid cartilage that is equal to about the head length of an anchor, or equal to about the distance from where a tissue manipulator exits a head, while holding the tissue manipulator at desired location. Alternatively, step 1022 can be omitted from method 1000. FIG. 36 illustrates the tissue manipulator 16 disposed through a window 1102 created in the thyroid cartilage 1104 and partially disposed within the arytenoid muscle 1106. The arytenoid muscle 1106 has been positioned in a desired location such that the vocal cord 1108 achieves the desired phonation. In addition, FIG. 36 illustrates a marking indicia 1110 disposed on the tissue manipulator 16.

Step 1026 can be accomplished by visualizing the thyroid cartilage and determining a suitable location near, or adjacent to, the window created in step 1004 that an anchor can be positioned. For example, any location that provides solid tissue or structure to which an anchor can be attached can utilized. Examples of locations considered suitable include locations that are positioned half way between a window created in the thyroid cartilage and the midline of the thyroid cartilage, such that the anchor is between about 1 millimeter and about 25 millimeters from a window created in the thyroid cartilage, and any other location considered suitable for a particular embodiment. An optional step comprises marking the location in which it is intended to position an anchor on the thyroid cartilage. This optional step can be accomplished using any suitable marking implement such as writing implements, pens, markers, and any other marking implement considered suitable for a particular embodiment.

Step 1028 can be accomplished as described herein with respect to step 824. For example, step 1028 can be accomplished by attaching the anchor to the thyroid cartilage at the desired location identified in step 1026. FIG. 37 illustrates an anchor 412 attached to the thyroid cartilage 1104 adjacent to the window 1102 created in the thyroid cartilage 1104.

An optional step that can be completed prior to step 1028 comprises creating one or more pilot holes through the thyroid cartilage sized and configured to receive a portion of a tissue fixation system and/or delivery tool (e.g., anchor 12, anchor 212, anchor 412). This optional step can be accomplished using any suitable drill and a drill bit that has a first outside diameter. A drill bit used complete any step of the methods described herein can have any suitable outside diameter, and skilled artisans will be able to select a suitable outside diameter for a drill bit according to a particular embodiment based on various considerations, including the structural arrangement at a point of treatment. Example outside diameters considered suitable for a drill bit include outside diameters that are equal to, less than, or greater than a shaft thread minor diameter. When a pilot hole is created, it is considered advantageous to use a drill bit with an outside diameter that is less than, or equal to, or about, a shaft thread minor diameter such that a friction fit can be accomplished between the anchor and the thyroid cartilage.

Step 1030 can be accomplished by applying an axial force on the shaft of the tissue manipulator toward, or away from, the thyroid cartilage until the marking indicia on the tissue manipulator is disposed adjacent the window. Alternatively, in embodiments in which a mark has not been applied to the tissue manipulator, step 1028 can alternatively comprise positioning the tissue manipulator adjacent to the window while asking a patient to phonate to confirm that the position of the tissue manipulator results in a desired phonation from the patient. Alternatively, step 1030 can be omitted from method 1000.

An optional step that can be completed subsequent to step 1030 comprises asking the patient to phonate. This optional step can be accomplished by providing instructions to a patient to phonate to determine whether placement of the vocal cord is satisfactory.

Step 1032 can be accomplished by applying an axial force on the shaft of the tissue manipulator orthogonal to the shaft lengthwise axis and toward the midline of the throat, or in any other direction, such that a bend is formed along the shaft between the new tissue manipulator end and the shaft second end. FIG. 37 illustrates the tissue manipulator 16 bent toward the midline of the throat. Depending on the anchor used in step 1026, step 1032 can be accomplished by positioning the tissue manipulator in the head second recess in embodiments in which anchor 212 or anchor 412 has been attached to the thyroid cartilage, or can be accomplished by positioning the tissue manipulator in the head third recess in embodiments in which anchor 12 has been attached to the thyroid cartilage.

Step 1034 can be accomplished as described herein with respect to step 834. An optional step that can be completed subsequent to step 1034 comprises asking the patient to phonate. This optional step can be accomplished by providing instructions to a patient to phonate to determine whether placement of the vocal cord is satisfactory.

Step 1036 can be accomplished as described herein with respect to step 828.

An optional that can be completed prior to, or in combination with, step 1036 comprises adjusting the position of the tissue manipulator. This optional step can be accomplished by applying an axial force toward, or away from, the thyroid cartilage on any suitable portion of the shaft of the tissue manipulator to position the arytenoid muscle in a desired location that can be based on the patient's anatomy or that results in a desired phonation. Alternatively, torque can be applied separately, or in combination with the axial force, to the shaft of the tissue manipulator (e.g., clockwise or counterclockwise direction about the shaft lengthwise axis) to achieved desired placement of vocal cord. Another optional step comprises maintaining the position of the tissue manipulator in the desired location while step 1036 is completed.

Step 1038 can be accomplished as described herein with respect to step 832. FIG. 38 illustrates an anchor 412 attached to the thyroid cartilage 1104 adjacent to the window 1102 created in the thyroid cartilage 1104 and the trimmed tissue manipulator disposed within the second passageway of the anchor 412.

Step 1040 can be accomplished as described herein with respect to step 826. Step 1042 can be accomplished as described herein with respect to step 838.

An optional step that can be completed prior to step 1042 comprises adding a bulking agent within the window. Any suitable bulking agent (e.g., collagen or gelatin based bulking agent) can be used and introduced into the window and selection of a suitable bulking agent can be based on various considerations, including the material forming a tissue manipulator. Alternatively, this optional step can be accomplished subsequent to step 1004.

While method 1000 has been described with respect to manipulating the position of an arytenoid muscle, any of the steps, alternative steps, and/or optional steps described herein can be utilized to treat any suitable tissue and a tissue fixation system can be attached to any suitable wall or feature of a patient. Selection of a suitable point of treatment to implant a tissue fixation system according to an embodiment can be based on various considerations, including the treatment intended to be performed and the structural arrangement at the treatment site.

While various steps, alternative steps, and optional steps have been described above with respect to the example method of treatment 1000 using a tissue fixation system and a delivery tool, these steps, alternative steps, and optional steps can be included in, accomplished concurrently with, and/or accomplished in the alternative to, the method steps, alternative steps, and/or optional steps described above with respect to the example method of treatment 800.

Those with ordinary skill in the art will appreciate that various modifications and alternatives for the described and illustrated embodiments can be developed in light of the overall teachings of the disclosure. Accordingly, the particular arrangements disclosed are intended to be illustrative only and not limiting as to the scope of the invention, which is to be given the full breadth of the appended claims and any and all equivalents thereof.

What is claimed is:

1. A tissue fixation system comprising:
an anchor having a shaft and a head attached to the shaft, the shaft having a shaft first end, a shaft second end, a shaft length extending from the shaft first end to the shaft second end, and a shaft main body defining a shaft outer surface, a shaft passageway extending from the shaft first end to the shaft second end, and a shaft thread extending from the shaft outer surface and along a portion of the shaft length, the head having a head first end attached to the shaft second end, a head second end, and a head main body defining a head outer surface, a head inner surface, a head first passageway, a head second passageway, a head third passageway, and a head thread, the head first passageway having a first inside diameter, extending from the head first end toward the head second end to the head second passageway, and in communication with the shaft passageway and the head second passageway, the head second passageway having a second inside diameter, extending from the head first passageway toward the head second end, and in communication with the head first passageway and the head third passageway, the head third passageway having a third inside diameter, extending from the head second passageway to the head outer surface, and in communication with the head second passageway, the first inside diameter being different than the second inside diameter, the second inside diameter being different than the third inside diameter, the head thread extending from the head inner surface and into the head third passageway;
a set screw partially disposed within the head third passageway, the set screw having a set screw first end, a set screw second end, a set screw length extending from the set screw first end to the set screw second end, and a set screw main body defining a set screw recess and a set screw thread extending along a portion of the set screw length, the set screw recess extending from the set screw second end toward the set screw first end, the set screw thread sized and configured to mate with the head thread, the set screw moveable within the head third passageway between a first position and a second position; and
a tissue manipulator partially disposed within the shaft passageway, the head first passageway, and the head second passageway, the tissue manipulator having a tissue manipulator shaft and a tissue manipulator coil attached to the tissue manipulator shaft, the tissue manipulator removable from the anchor when the set screw is in the first position and releasably attached to the anchor when the set screw is in the second position.

2. The tissue fixation system of claim 1, wherein the head second passageway has a head second passageway lengthwise axis; and
wherein the head third passageway has a head third passageway lengthwise axis that is disposed at an angle between about 0 degrees and about 180 degrees relative to the head second passageway lengthwise axis.

3. The tissue fixation system of claim 2, wherein the angle is about 90 degrees.

4. The tissue fixation system of claim 1, wherein the shaft has a shaft outside diameter; and
wherein the head third passageway has a head third passageway inside diameter that is greater than the shaft outside diameter.

5. The tissue fixation system of claim 1, wherein the head comprises a hexagonal cross-sectional configuration.

6. The tissue fixation system of claim 1, wherein the set screw has a set screw outer surface and the set screw thread extends from the set screw outer surface.

7. The tissue fixation system of claim 1, wherein the head second passageway extends from the head first passageway to the head second end.

8. The tissue fixation system of claim 1, wherein the head second passageway is sized and configured to receive a portion of the tissue manipulator.

9. The tissue fixation system of claim 1, wherein the set screw recess is sized and configured to receive a portion of a hand tool.

10. The tissue fixation system of claim 1, wherein the head has a head outside diameter; and
wherein the head second passageway has a head second passageway inside diameter that is equal to about the head outside diameter.

11. The tissue fixation system of claim 1, wherein the shaft thread is self-tapping.

12. A tissue fixation system comprising:
an anchor having a shaft and a head attached to the shaft, the shaft having a shaft first end, a shaft second end, a shaft length extending from the shaft first end to the shaft second end, and a shaft main body defining a shaft outer surface, a shaft passageway extending from the shaft first end to the shaft second end, and a shaft thread extending from the shaft outer surface and along a portion of the shaft length, the head having a hexagonal cross-sectional configuration, a head first end attached to the shaft second end, a head second end, and a head main body defining a head outer surface, a head inner surface, a head first passageway, a head second passageway, a head third passageway, and a head thread, the head first passageway having a first inside diameter, extending from the head first end toward the head second end to the head second passageway, and in communication with the shaft passageway and the head second passageway, the head second passageway having a head second passageway lengthwise axis, a second inside diameter, extending from the head first passageway toward the head second end, and in communication with the head first passageway and the head third passageway, the head third passageway having a head third passageway lengthwise axis that is disposed at an angle about 90 degrees relative to the head second passageway lengthwise axis, a third inside diameter, extending from the head second passageway to the head outer surface, and in communication with the head second passageway, the first inside diameter being different than the second inside diameter, the second inside diameter being different than the third inside diameter, the head thread extending from the head inner surface and into the head third passageway;
a set screw partially disposed within the head third passageway, the set screw having a set screw first end, a set screw second end, a set screw length extending from the set screw first end to the set screw second end, and a set screw main body defining a set screw recess and a set screw thread extending along a portion of the set screw length, the set screw recess extending from the set screw second end toward the set screw first end, the set screw thread sized and configured to mate with the head thread, the set screw moveable within the head third passageway between a first position and a second position; and
a tissue manipulator partially disposed within the shaft passageway, the head first passageway, and the head second passageway, the tissue manipulator having a tissue manipulator shaft and a tissue manipulator coil attached to the tissue manipulator shaft, the tissue manipulator removable from the anchor when the set screw is in the first position and releasably attached to the anchor when the set screw is in the second position.

13. The tissue fixation system of claim 12, wherein the shaft has a shaft outside diameter; and
wherein the head third passageway has a head third passageway inside diameter that is greater than the shaft outside diameter.

14. The tissue fixation system of claim 12, wherein the set screw has a set screw outer surface and the set screw thread extends from the set screw outer surface.

15. The tissue fixation system of claim 12, wherein the head second passageway extends from the head first passageway to the head second end.

16. The tissue fixation system of claim 12, wherein the head second passageway is sized and configured to receive a portion of the tissue manipulator.

17. The tissue fixation system of claim 12, wherein the set screw recess is sized and configured to receive a portion of a hand tool.

18. The tissue fixation system of claim 12, wherein the head has a head outside diameter; and
wherein the head second passageway has a head second passageway inside diameter that is equal to about the head outside diameter.

19. The tissue fixation system of claim 12, wherein the shaft thread is self-tapping.

20. A tissue fixation system comprising:
an anchor having a shaft and a head attached to the shaft, the shaft having a shaft first end, a shaft second end, a shaft length extending from the shaft first end to the shaft second end, and a shaft main body defining a shaft outer surface, a shaft passageway extending from the shaft first end to the shaft second end, and a self-tapping shaft thread extending from the shaft outer surface and along a portion of the shaft length, the head having a hexagonal cross-sectional configuration, a head first end attached to the shaft second end, a head second end, and a head main body defining a head outer surface, a head inner surface, a head first passageway, a head second passageway, a head third passageway, and a head thread, the head first passageway having a first inside diameter, extending from the head first end toward the head second end to the head second passageway, and in communication with the shaft passageway and the head second passageway, the head second passageway having a head second passageway lengthwise axis, a second inside diameter, extending from the head first passageway to the head second end, and in communication with the head first passageway and the head third passageway, the head third passageway having a head third passageway lengthwise axis that is disposed at an angle about 90 degrees relative to the head second passageway lengthwise axis, a third inside diameter, extending from the head second passageway to the head outer surface, and in communication with the head second passageway, the first inside diameter being different than the second inside diameter, the second inside diameter being different than the third inside diameter, the head thread extending from the head inner surface and into the head third passageway;
a set screw partially disposed within the head third passageway, the set screw having a set screw first end, a set screw second end, a set screw length extending from the set screw first end to the set screw second end, and a set screw main body defining a set screw recess and a set screw thread extending along a portion of the set screw length, the set screw recess extending from the set screw second end toward the set screw first end, the set screw thread sized and configured to mate with the head thread, the set screw moveable within the head third passageway between a first position and a second position; and
a tissue manipulator partially disposed within the shaft passageway, the head first passageway, and the head second passageway, the tissue manipulator having a tissue manipulator shaft and a tissue manipulator coil attached to the tissue manipulator shaft, the tissue manipulator removable from the anchor when the set screw is in the first position and releasably attached to the anchor when the set screw is in the second position;
wherein the head second passageway is sized and configured to receive a portion of the tissue manipulator.
